(19) United States
(12) Patent Application Publication
(10) Pub. No.: US 2024/0150840 A1
(43) Pub. Date: May 9, 2024

(54) INTRA-INDIVIDUAL ANALYSIS FOR PRESENCE OF HEALTH CONDITIONS

(71) Applicant: Flagship Pioneering Innovations VI, LLC, Cambridge, MA (US)

(72) Inventor: Anthony P. Shuber, Cambridge, MA (US)

(21) Appl. No.: 18/173,049

(22) Filed: Feb. 22, 2023

Related U.S. Application Data
(60) Provisional application No. 63/432,006, filed on Dec. 12, 2022.
(60) Provisional application No. 63/312,741, filed on Feb. 22, 2022.

(51) Int. Cl. C12Q 1/6886; C12Q 1/6858

(52) U.S. Cl. CPC C12Q 1/6886; C12Q 1/6858

(57) ABSTRACT

Disclosed herein are methods, non-transitory computer readable media, systems, and kits for performing an intra-individual analysis for determining presence or absence of a health condition in an individual. Specifically, the intra-individual analysis involves combining sequence information from target nucleic acids with sequence information from reference nucleic acids obtained from the individual. The target nucleic acids include signatures that may be informative for determining presence or absence of the health condition and the reference nucleic acids include baseline biological signatures of the individual. By combining sequence information from the target nucleic acids and the reference nucleic acids, the resulting generated signal is more informative for determining presence or absence of the health condition in comparison to sequence information of the target nucleic acids alone.

### CROSS-REFERENCE TO RELATED APPLICATIONS

This application claims the benefit of and priority to U.S. Provisional Patent Application No. 63/312,741 filed Feb. 22, 2022 and U.S. Provisional Patent Application No. 63/432,006 filed Dec. 12, 2022, the entire disclosure of each of which is hereby incorporated by reference in its entirety for all purposes.

### SEQUENCE LISTING

The instant application contains a Sequence Listing which has been submitted electronically in XML format via EFS-Web and is hereby incorporated by reference in its entirety. Said XML copy, created on Aug. 17, 2023, is named FLG-013US_SL.xml and is 776,548 bytes in size.

### BACKGROUND

Conventional detection methods involve analyzing a wealth of information to determine presence of a disease in a patient. However, not all information may be relevant or informative. Including such information in the analysis can have a confounding effect and therefore, are detrimental towards the final predictive accuracy. Thus, there is a need to eliminate non-informative signatures to improve predictive accuracy.

### SUMMARY

Disclosed herein are methods for performing an individual-specific analysis, hereafter referred to as an intra-individual analysis, for improved detection of a signal present in a sample obtained from the individual. In various embodiments, such a signal is informative for determining presence or absence of a health condition in the individual. The intra-individual analysis removes baseline biological signatures of the individual which are less informative or not informative of presence of absence of a health condition. By eliminating baseline biological signatures, the remaining signatures are used to more accurately predict presence or absence of a health condition in the individual. Specifically, the intra-individual analysis involves combining sequence information from target nucleic acids with sequence information from reference nucleic acids obtained from the individual. The target nucleic acids include signatures that are informative for determining presence or absence of the health condition and the reference nucleic acids include baseline biological signatures of the individual. By combining sequence information from the target nucleic acids and the reference nucleic acids, the resulting combined signal is more informative for determining presence or absence of the health condition in comparison to sequence information of the target nucleic acids alone.

Disclosed herein is a method for determining a signal informative of a health condition from an individual, the method comprising: obtaining target nucleic acids and reference nucleic acids from one or more samples from the individual; generating sequence information from the target nucleic acids and sequence information from the reference nucleic acids; and combining the sequence information from the target nucleic acids and the sequence information from the reference nucleic acids to generate the signal informative of the health condition. In various embodiments, the health condition is a cancer. In various embodiments, the health condition is an early stage cancer or preclinical phase cancer.

In various embodiments, obtaining target nucleic acids and reference nucleic acids from one or more samples comprises obtaining the target nucleic acids and the reference nucleic acids from a single sample. In various embodiments, the single sample is any one of a blood sample, a stool sample, a urine sample, a mucous sample, or a saliva sample. In various embodiments, obtaining target nucleic acids and reference nucleic acids comprises fractionating the single sample, wherein the target nucleic acids are obtained from a first fraction of the single sample, and wherein the reference nucleic acids are obtained from a second fraction of the single sample. In various embodiments, the target nucleic acids comprise cell free DNA (cfDNA). In various embodiments, the reference nucleic acids comprise genomic DNA from cells of the individual. In various embodiments, the cells of the individual comprise peripheral blood mononuclear cells (PBMCs) or polymorphonuclear cells.

In various embodiments, obtaining target nucleic acids and reference nucleic acids from one or more samples comprises obtaining the target nucleic acids and the reference nucleic acids from different samples. In various embodiments, the target nucleic acids are obtained from a blood sample, and wherein the reference nucleic acids are obtained from a tissue sample. In various embodiments, the target nucleic acids comprise cell free DNA (cfDNA). In various embodiments, the reference nucleic acids comprise genomic DNA from cells of the individual. In various embodiments, combining the sequence information from the target nucleic acids and the sequence information from the reference nucleic acids comprises aligning the sequence information from the target nucleic acids and the sequence information from the reference nucleic acids. In various embodiments, combining the sequence information from the target nucleic acids and the sequence information from the reference nucleic acids comprises determining a difference between the sequence information from the target nucleic acids and the sequence information from the reference nucleic acids. In various embodiments, combining the sequence information from the target nucleic acids and the sequence information from the reference nucleic acids comprises subtracting the sequence information from the reference nucleic acids from the sequence information from the target nucleic acids. In various embodiments, the sequence information from the target nucleic acids comprises methylation sequence information of the target nucleic acids.

In various embodiments, the sequence information from the target nucleic acids comprises phased sequencing information of the target nucleic acids. In various embodiments, the phased sequence information from the target nucleic acids comprises sequencing information derived from one of two or more sources. In various embodiments, the phased sequence information from the target nucleic acids is generated by: aligning sequence reads of target nucleic acids to long sequence reads of reference nucleic acids to determine two or more sources of the target nucleic acids, wherein the long sequence reads of reference nucleic acids comprise at least 500 bases; and categorizing target nucleic acids derived from one of the two or more sources. In various embodiments, the long sequence reads of reference nucleic acids comprise at least 500 bases, at least 1000 bases, at least 2000 bases, at least 3000 bases, at least 4000 bases, at least 5000 bases, at least 6000 bases, at least 7000 bases, at least 8000 bases, at least 9000, at least 10,000 bases, at least 12,000 bases, at least 15,000 bases, at least 20,000 bases, at least 25,000 bases, at least 30,000 bases, at least 40,000 bases, at least 50,000 bases, at least 60,000 bases, at least 70,000 bases, at least 80,000 bases, at least 90,000 bases, or at least 100,000 bases. In various embodiments, the long sequence reads of reference nucleic acids comprise between 5,000 bases and 100,000 bases. In various embodiments, the two or more sources comprise a maternal chromosome and a paternal chromosome.

In various embodiments, the sequence information from the reference nucleic acids comprises methylation sequence information of the reference nucleic acids. In various embodiments, the methylation sequence information of the target nucleic acids and the methylation sequence information of the reference nucleic acids both comprise methylation statuses for a plurality of genomic sites. In various embodiments, the plurality of genomic sites comprise one or more CpG islands or portions of CpG islands shown in Tables 1-4.

In various embodiments, generating sequence information from the target nucleic acids and sequence information from the reference nucleic acids comprises performing an assay, wherein the assay comprises one or more of a. sequencing of target nucleic acids and/or reference nucleic acids via targeted sequencing, whole genome sequencing, or whole genome bisulfite sequencing; b. shallow sequencing and/or deep sequencing; c. a nucleic acid amplification assay; and d. an assay that generates methylation information. In various embodiments, performing the assay comprises performing both shallow sequencing and deep sequencing. In various embodiments, performing both shallow sequencing and deep sequencing comprises: performing shallow sequencing to generate sequence information from the reference nucleic acids; and performing deep sequencing to generate sequence information from the target nucleic acids. In various embodiments, performing shallow sequencing comprises generating less than less than 50 reads per base, less than 40 reads per base, less than 30 reads per base, less than 20 reads per base, less than 10 reads per base, less than 9 reads per base, less than 8 reads per base, less than 7 reads per base, less than 6 reads per base, or less than 5 reads per base. In various embodiments, performing deep sequencing comprises generating greater than 50 reads per base, greater than 60 reads per base, greater than 70 reads per base, greater than 80 reads per base, greater than 90 reads per base, greater than 100 reads per base, greater than 120 reads per base, greater than 140 reads per base, greater than 150 reads per base, greater than 170 reads per base, greater than 200 reads per base, greater than 225 reads per base, greater than 250 reads per base, greater than 300 reads per base, greater than 400 reads per base, or greater than 500 reads per base.

In various embodiments, the nucleic acid amplification assay is a PCR assay. In various embodiments, the PCR assay comprises a real-time PCR assay, quantitative real-time PCR (qPCR) assay, digital PCR (dPCR) assay, allele-specific PCR assay, or reverse-transcription PCR assay. In various embodiments, generating sequence information from the target nucleic acids and sequence information from the reference nucleic acids comprises performing a target enrichment assay. In various embodiments, the target enrichment assay comprises hybrid capture. In various embodiments, performing the assay comprises: obtaining bisulfite converted target nucleic acids and/or reference nucleic acids; and selectively amplifying target regions of the bisulfite converted target nucleic acids and/or reference nucleic acids. In various embodiments, performing the assay further comprises: determining quantitative values of sequences of the amplicons comprising the amplified target regions to generate the sequence information of the target nucleic acids and/or sequence information of the reference nucleic acids. In various embodiments, the quantitative values comprise cycle threshold (Ct) values. In various embodiments, performing the assay further comprises: sequencing amplicons comprising the amplified target regions to generate the sequence information of the target nucleic acids and/or sequence information of the reference nucleic acids. In various embodiments, the target regions comprise previously identified regions that are differentially methylated in presence of the health condition. In various embodiments, the target regions comprise one or more CpG islands or portions of CpG islands shown in Tables 1-4.

In various embodiments, methods disclosed herein further comprise: determining a tissue of origin of the health condition using the signal informative of the health condition. In various embodiments, methods disclosed herein further comprise: determining progression of the health condition using the signal informative of the health condition.

In various embodiments, combining the sequence information from the target nucleic acids and the sequence information from the reference nucleic acids comprises determining ratios of methylation levels amongst two or more genomic sites from the target nucleic acids. In various embodiments, the two or more genomic sites are on a common CpG island. In various embodiments, the two or more genomic sites are on different CpG islands. In various embodiments, a subset of the two or more CpG sites are in a common CpG island, and a second subset of the two or more CpG sites are in at least a different CpG island. In various embodiments, combining the sequence information from the target nucleic acids and the sequence information from the reference nucleic acids further comprises determining a difference between the sequence information from target nucleic acids and sequence information from reference nucleic acids to generate a signal that includes limited or no baseline signatures. In various embodiments, combining the sequence information from the target nucleic acids and the sequence information from the reference nucleic acids further comprises determining additional ratios of methylation levels amongst the two or more CpG sites from the signal that includes limited or no baseline signatures. In various embodiments, combining the sequence information from the target nucleic acids and the sequence information from the reference nucleic acids further comprises comparing the ratios of methylation levels amongst two or more CpG sites generated from target nucleic acids and the additional ratios of methylation levels amongst the two or more CpG sites generated from the signal that includes limited or no baseline signatures. In various embodiments, methods disclosed herein further comprise generating a prediction of presence or absence of the health condition based on the comparison. In various embodiments, if the comparison yields no change between the ratios and the additional ratios, then the generated prediction comprises absence of the health condition. In various embodiments, if the comparison yields a change between the ratios and the additional ratios, then the generated prediction comprises presence of the health condition. In various embodiments, the two or more CpG sites are located in CpG islands or portions of CpG islands shown in Tables 1-4.

Additionally disclosed herein is a method of identifying a cancer signal from an individual, the method comprising: obtaining a sample from the individual, wherein the sample comprises cfDNA and a PBMC DNA; determining the methylation status at a plurality of CpG sites of the cfDNA and the PBMC DNA; and comparing the methylation status at the plurality of CpG sites of the cfDNA and the PBMC DNA to generate the signal informative of the health condition. In various embodiments, the methylation status was determined from sequencing or nucleic acid amplification. In various embodiments, the nucleic acid amplification comprises a PCR assay. In various embodiments, the PCR assay comprises a real-time PCR assay, quantitative real-time PCR (qPCR) assay, digital PCR (dPCR) assay, allele-specific PCR assay, or reverse-transcription PCR assay. In various embodiments, the CPG sites comprise previously identified CPG sites that are differentially methylated in presence of the health condition. In various embodiments, the CpG sites comprise one or more CpG islands or portions of CpG islands shown in Tables 1-4. In various embodiments, determining the methylation status at a plurality of CpG sites of the cfDNA and the PBMC DNA comprises: aligning sequence reads of the cfDNA to long sequence reads of the PBMC DNA to determine two or more sources of the cfDNA, wherein the long sequence reads of the PBMC DNA comprise at least 500 bases; and categorizing cfDNA as being derived from one of the two or more sources. In various embodiments, the long sequence reads of reference nucleic acids comprise at least 500 bases, at least 1000 bases, at least 2000 bases, at least 3000 bases, at least 4000 bases, at least 5000 bases, at least 6000 bases, at least 7000 bases, at least 8000 bases, at least 9000, at least 10,000 bases, at least 12,000 bases, at least 15,000 bases, at least 20,000 bases, at least 25,000 bases, at least 30,000 bases, at least 40,000 bases, at least 50,000 bases, at least 60,000 bases, at least 70,000 bases, at least 80,000 bases, at least 90,000 bases, or at least 100,000 bases. In various embodiments, the long sequence reads of reference nucleic acids comprise between 5,000 bases and 30,000 bases. In various embodiments, the two or more sources comprise a maternal chromosome and a paternal chromosome.

Additionally disclosed herein is a non-transitory computer readable medium comprising instructions that, when executed by a processor, cause the processor to: generate sequence information from target nucleic acids and sequence information from reference nucleic acids, wherein the target nucleic acids and reference nucleic acids are obtained from one or more samples from an individual; and combine the sequence information from the target nucleic acids and the sequence information from the reference nucleic acids to generate the signal informative of the health condition. In various embodiments, the health condition is a cancer. In various embodiments, the health condition is an early stage cancer or preclinical phase cancer. In various embodiments, the target nucleic acids and reference nucleic acids are obtained from a single sample. In various embodiments, the single sample is any one of a blood sample, a stool sample, a urine sample, a mucous sample, or a saliva sample. In various embodiments, the single sample previously underwent fractionation, wherein the target nucleic acids are obtained from a first fraction of the single sample, and wherein the reference nucleic acids are obtained from a second fraction of the single sample. In various embodiments, the target nucleic acids comprise cell free DNA (cfDNA). In various embodiments, the reference nucleic acids comprise genomic DNA from cells of the individual. In various embodiments, the cells of the individual comprise peripheral blood mononuclear cells (PBMCs) or polymorphonuclear cells. In various embodiments, the target nucleic acids and reference nucleic acids are obtained from different samples. In various embodiments, the target nucleic acids are obtained from a blood sample, and wherein the reference nucleic acids are obtained from a tissue sample. In various embodiments, the target nucleic acids comprise cell free DNA (cfDNA). In various embodiments, the reference nucleic acids comprise genomic DNA from cells of the individual.

In various embodiments, the instructions that cause the processor to combine the sequence information from the target nucleic acids and the sequence information from the reference nucleic acids further comprises instructions that, when executed by the processor, cause the processor to align the sequence information from the target nucleic acids and the sequence information from the reference nucleic acids. In various embodiments, the instructions that cause the processor to combine the sequence information from the target nucleic acids and the sequence information from the reference nucleic acids further comprises instructions that, when executed by the processor, cause the processor to determine a difference between the sequence information from the target nucleic acids and the sequence information from the reference nucleic acids. In various embodiments, the instructions that cause the processor to combine the sequence information from the target nucleic acids and the sequence information from the reference nucleic acids further comprises instructions that, when executed by the processor, cause the processor to subtract the sequence information from the reference nucleic acids from the sequence information from the target nucleic acids.

In various embodiments, the sequence information from the target nucleic acids comprises methylation sequence information of the target nucleic acids. In various embodiments, the sequence information from the target nucleic acids comprises phased sequencing information from the target nucleic acids. In various embodiments, the phased sequence information of the target nucleic acids comprises sequencing information derived from one of two or more sources. In various embodiments, the phased sequence information from the target nucleic acids is generated by: aligning sequence reads of target nucleic acids to long sequence reads of reference nucleic acids to determine two or more sources of the target nucleic acids, wherein the long sequence reads of reference nucleic acids comprise at least 500 bases; and categorizing target nucleic acids derived from one of the two or more sources. In various embodiments, the long sequence reads of reference nucleic acids comprise at least 500 bases, at least 1000 bases, at least 2000 bases, at least 3000 bases, at least 4000 bases, at least 5000 bases, at least 6000 bases, at least 7000 bases, at least 8000 bases, at least 9000, at least 10,000 bases, at least 12,000 bases, at least 15,000 bases, at least 20,000 bases, at least 25,000 bases, at least 30,000 bases, at least 40,000 bases, at least 50,000 bases, at least 60,000 bases, at least 70,000 bases, at least 80,000 bases, at least 90,000 bases, or at least 100,000 bases. In various embodiments, the long sequence reads of reference nucleic acids comprise between 5,000 bases and 100,000 bases. In various embodiments, the two or more sources comprise a maternal chromosome and a paternal chromosome. In various embodiments, the sequence information from the reference nucleic acids comprises methylation sequence information from the reference nucleic acids. In various embodiments, the methylation sequence information of the target nucleic acids and the methylation sequence information of the reference nucleic acids both comprise methylation statuses for a plurality of genomic sites. In various embodiments, the plurality of genomic sites comprise one or more CpG islands or portions of CpG islands shown in Tables 1-4.

In various embodiments, the sequence information from target nucleic acids is generated from shallow sequencing, and wherein the sequence information from reference nucleic acids is generated from deep sequencing. In various embodiments, shallow sequencing comprises generating less than less than 50 reads per base, less than 40 reads per base, less than 30 reads per base, less than 20 reads per base, less than 10 reads per base, less than 9 reads per base, less than 8 reads per base, less than 7 reads per base, less than 6 reads per base, or less than 5 reads per base. In various embodiments, deep sequencing comprises generating greater than 50 reads per base, greater than 60 reads per base, greater than 70 reads per base, greater than 80 reads per base, greater than 90 reads per base, greater than 100 reads per base, greater than 120 reads per base, greater than 140 reads per base, greater than 150 reads per base, greater than 170 reads per base, greater than 200 reads per base, greater than 225 reads per base, greater than 250 reads per base, greater than 300 reads per base, greater than 400 reads per base, or greater than 500 reads per base. In various embodiments, the non-transitory computer readable medium further comprises instructions that, when executed by a processor, cause the processor to: determine a tissue of origin of the health condition using the signal informative of the health condition. In various embodiments, the non-transitory computer readable medium further comprises instructions that, when executed by a processor, cause the processor to: determine progression of the health condition using the signal informative of the health condition.

In various embodiments, combining the sequence information from the target nucleic acids and the sequence information from the reference nucleic acids comprises determining ratios of methylation levels amongst two or more genomic sites from the target nucleic acids. In various embodiments, the two or more genomic sites are on a common CpG island. In various embodiments, the two or more genomic sites are on different CpG islands. In various embodiments, a subset of the two or more CpG sites are in a common CpG island, and a second subset of the two or more CpG sites are in at least a different CpG island. In various embodiments, combining the sequence information from the target nucleic acids and the sequence information from the reference nucleic acids further comprises determining a difference between the sequence information from target nucleic acids and sequence information from reference nucleic acids to generate a signal that includes limited or no baseline signatures. In various embodiments, combining the sequence information from the target nucleic acids and the sequence information from the reference nucleic acids further comprises determining additional ratios of methylation levels amongst the two or more CpG sites from the signal that includes limited or no baseline signatures. In various embodiments, combining the sequence information from the target nucleic acids and the sequence information from the reference nucleic acids further comprises comparing the ratios of methylation levels amongst two or more CpG sites generated from target nucleic acids and the additional ratios of methylation levels amongst the two or more CpG sites generated from the signal that includes limited or no baseline signatures. In various embodiments, methods disclosed herein further comprise generating a prediction of presence or absence of the health condition based on the comparison. In various embodiments, if the comparison yields no change between the ratios and the additional ratios, then the generated prediction comprises absence of the health condition. In various embodiments, if the comparison yields a change between the ratios and the additional ratios, then the generated prediction comprises presence of the health condition. In various embodiments, the two or more CpG sites are located in CpG islands or portions of CpG islands shown in Tables 1-4.

Additionally disclosed herein is a system comprising: a processor; a data storage comprising sequence information from target nucleic acids and sequence information from reference nucleic acids, wherein the target nucleic acids and reference nucleic acids are obtained from one or more samples from an individual; a non-transitory computer readable medium comprising instructions that, when executed by the processor, cause the processor to: combine the sequence information from the target nucleic acids and the sequence information from the reference nucleic acids to generate the signal informative of the health condition. In various embodiments, the health condition is a cancer. In various embodiments, the health condition is an early stage cancer or preclinical phase cancer. In various embodiments, the target nucleic acids and reference nucleic acids are obtained from a single sample. In various embodiments, the single sample is any one of a blood sample, a stool sample, a urine sample, a mucous sample, or a saliva sample. In various embodiments, the single sample previously underwent fractionation, wherein the target nucleic acids are obtained from a first fraction of the single sample, and wherein the reference nucleic acids are obtained from a second fraction of the single sample. In various embodiments, the target nucleic acids comprise cell free DNA (cfDNA). In various embodiments, the reference nucleic acids comprise genomic DNA from cells of the individual. In various embodiments, the cells of the individual comprise peripheral blood mononuclear cells (PBMCs) or polymorphonuclear cells. In various embodiments, the target nucleic acids and reference nucleic acids are obtained from different samples. In various embodiments, the target nucleic acids are obtained from a blood sample, and wherein the reference nucleic acids are obtained from a tissue sample. In various embodiments, the target nucleic acids comprise cell free DNA (cfDNA). In various embodiments, the reference nucleic acids comprise genomic DNA from cells of the individual.

In various embodiments, the instructions that cause the processor to combine the sequence information from the target nucleic acids and the sequence information from the reference nucleic acids further comprises instructions that, when executed by the processor, cause the processor to align the sequence information from the target nucleic acids and the sequence information from the reference nucleic acids. In various embodiments, the instructions that cause the processor to combine the sequence information from the target nucleic acids and the sequence information from the reference nucleic acids further comprises instructions that, when executed by the processor, cause the processor to determine a difference between the sequence information from the target nucleic acids and the sequence information from the reference nucleic acids. In various embodiments, the instructions that cause the processor to combine the sequence information from the target nucleic acids and the sequence information from the reference nucleic acids further comprises instructions that, when executed by the processor, cause the processor to subtract the sequence information of the reference nucleic acids from the sequence information of the target nucleic acids. In various embodiments, the sequence information from the target nucleic acids comprises methylation sequence information of the target nucleic acids. In various embodiments, the sequence information from the target nucleic acids comprises phased sequencing information of the target nucleic acids. In various embodiments, the phased sequence information from the target nucleic acids comprises sequencing information derived from one of two or more sources. In various embodiments, the phased sequence information from the target nucleic acids is generated by: aligning sequence reads of target nucleic acids to long sequence reads of reference nucleic acids to determine two or more sources of the target nucleic acids, wherein the long sequence reads of reference nucleic acids comprise at least 500 bases; and categorizing target nucleic acids as being derived from one of the two or more sources. In various embodiments, the long sequence reads of reference nucleic acids comprise at least 500 bases, at least 1000 bases, at least 2000 bases, at least 3000 bases, at least 4000 bases, at least 5000 bases, at least 6000 bases, at least 7000 bases, at least 8000 bases, at least 9000, at least 10,000 bases, at least 12,000 bases, at least 15,000 bases, at least 20,000 bases, at least 25,000 bases, at least 30,000 bases, at least 40,000 bases, at least 50,000 bases, at least 60,000 bases, at least 70,000 bases, at least 80,000 bases, at least 90,000 bases, or at least 100,000 bases. In various embodiments, the long sequence reads of reference nucleic acids comprise between 5,000 bases and 100,000 bases. In various embodiments, the two or more sources comprise a maternal chromosome and a paternal chromosome

In various embodiments, the sequence information from the reference nucleic acids comprises methylation sequence information of the reference nucleic acids. In various embodiments, the methylation sequence information of the target nucleic acids and the methylation sequence information of the reference nucleic acids both comprise methylation statuses for a plurality of genomic sites. In various embodiments, the plurality of genomic sites comprise one or more CpG islands or portions of CpG islands shown in Tables 1-4.

In various embodiments, the sequence information from target nucleic acids is generated from shallow sequencing, and wherein the sequence information from reference nucleic acids is generated from deep sequencing. In various embodiments, shallow sequencing comprises generating less than less than 50 reads per base, less than 40 reads per base, less than 30 reads per base, less than 20 reads per base, less than 10 reads per base, less than 9 reads per base, less than 8 reads per base, less than 7 reads per base, less than 6 reads per base, or less than 5 reads per base. In various embodiments, deep sequencing comprises generating greater than 50 reads per base, greater than 60 reads per base, greater than 70 reads per base, greater than 80 reads per base, greater than 90 reads per base, greater than 100 reads per base, greater than 120 reads per base, greater than 140 reads per base, greater than 150 reads per base, greater than 170 reads per base, greater than 200 reads per base, greater than 225 reads per base, greater than 250 reads per base, greater than 300 reads per base, greater than 400 reads per base, or greater than 500 reads per base.

In various embodiments, the non-transitory computer readable medium further comprises instructions that, when executed by a processor, cause the processor to: determine a tissue of origin of the health condition using the signal informative of the health condition. In various embodiments, the non-transitory computer readable medium further comprises instructions that, when executed by a processor, cause the processor to: determine progression of the health condition using the signal informative of the health condition.

In various embodiments, combining the sequence information from the target nucleic acids and the sequence information from the reference nucleic acids comprises determining ratios of methylation levels amongst two or more genomic sites from the target nucleic acids. In various embodiments, the two or more genomic sites are on a common CpG island. In various embodiments, the two or more genomic sites are on different CpG islands. In various embodiments, a subset of the two or more CpG sites are in a common CpG island, and a second subset of the two or more CpG sites are in at least a different CpG island. In various embodiments, combining the sequence information from the target nucleic acids and the sequence information from the reference nucleic acids further comprises determining a difference between the sequence information from target nucleic acids and sequence information from reference nucleic acids to generate a signal that includes limited or no baseline signatures. In various embodiments, combining the sequence information from the target nucleic acids and the sequence information from the reference nucleic acids further comprises determining additional ratios of methylation levels amongst the two or more CpG sites from the signal that includes limited or no baseline signatures. In various embodiments, combining the sequence information from the target nucleic acids and the sequence information from the reference nucleic acids further comprises comparing the ratios of methylation levels amongst two or more CpG sites generated from target nucleic acids and the additional ratios of methylation levels amongst the two or more CpG sites generated from the signal that includes limited or no baseline signatures. In various embodiments, methods disclosed herein further comprise generating a prediction of presence or absence of the health condition based on the comparison. In various embodiments, if the comparison yields no change between the ratios and the additional ratios, then the generated prediction comprises absence of the health condition. In various embodiments, if the comparison yields a change between the ratios and the additional ratios, then the generated prediction comprises presence of the health condition. In various embodiments, the two or more CpG sites are located in CpG islands or portions of CpG islands shown in Tables 1-4.

Additionally disclosed herein is a kit comprising: a. equipment to draw one or more samples from an individual; b. a set of detection reagents for generating sequence information for target nucleic acids and sequence information for reference nucleic acids in the one or more samples; and c. instructions for accessing computer program instructions stored on a computer storage medium that, when executed by a processor of a computer system, cause the processor to: combine the sequence information from the target nucleic acids and the sequence information from the reference nucleic acids to generate the signal informative of the health condition. In various embodiments, the health condition is a cancer. In various embodiments, the health condition is an early stage cancer or preclinical phase cancer. In various embodiments, the target nucleic acids and reference nucleic acids are obtained from a single sample. In various embodiments, the single sample is any one of a blood sample, a stool sample, a urine sample, a mucous sample, or a saliva sample. In various embodiments, the single sample was previously fractionated, wherein the target nucleic acids are obtained from a first fraction of the single sample, and wherein the reference nucleic acids are obtained from a second fraction of the single sample. In various embodiments, the target nucleic acids comprise cell free DNA (cfDNA). In various embodiments, the reference nucleic acids comprise genomic DNA from cells of the individual. In various embodiments, the cells of the individual comprise peripheral blood mononuclear cells (PBMCs) or polymorphonuclear cells. In various embodiments, the target nucleic acids and reference nucleic acids are obtained from different samples. In various embodiments, the target nucleic acids are obtained from a blood sample, and wherein the reference nucleic acids are obtained from a tissue sample. In various embodiments, the target nucleic acids comprise cell free DNA (cfDNA). In various embodiments, the reference nucleic acids comprise genomic DNA from cells of the individual.

In various embodiments, the computer program instructions that cause the processor to combine the sequence information from the target nucleic acids and the sequence information from the reference nucleic acids further comprises instructions that, when executed by the processor, cause the processor to align the sequence information from the target nucleic acids and the sequence information from the reference nucleic acids. In various embodiments, the computer program instructions that cause the processor to combine the sequence information from the target nucleic acids and the sequence information from the reference nucleic acids further comprises instructions that, when executed by the processor, cause the processor to determine a difference between the sequence information from the target nucleic acids and the sequence information from the reference nucleic acids. In various embodiments, the computer program instructions that cause the processor to combine the sequence information from the target nucleic acids and the sequence information from the reference nucleic acids further comprises instructions that, when executed by the processor, cause the processor to subtract the sequence information of the reference nucleic acids from the sequence information of the target nucleic acids.

In various embodiments, the sequence information from the target nucleic acids comprises methylation sequence information of the target nucleic acids. In various embodiments, the sequence information from the target nucleic acids comprises phased sequencing information from the target nucleic acids. In various embodiments, the phased sequence information of the target nucleic acids comprises sequencing information derived from one of two or more sources. In various embodiments, the phased sequence information from the target nucleic acids is generated by: aligning sequence reads of target nucleic acids to long sequence reads of reference nucleic acids to determine two or more sources of the target nucleic acids, wherein the long sequence reads of reference nucleic acids comprise at least 500 bases; and categorizing target nucleic acids as being derived from one of the two or more sources. In various embodiments, the long sequence reads of reference nucleic acids comprise at least 500 bases, at least 1000 bases, at least 2000 bases, at least 3000 bases, at least 4000 bases, at least 5000 bases, at least 6000 bases, at least 7000 bases, at least 8000 bases, at least 9000, at least 10,000 bases, at least 12,000 bases, at least 15,000 bases, at least 20,000 bases, at least 25,000 bases, at least 30,000 bases, at least 40,000 bases, at least 50,000 bases, at least 60,000 bases, at least 70,000 bases, at least 80,000 bases, at least 90,000 bases, or at least 100,000 bases. In various embodiments, the long sequence reads of reference nucleic acids comprise between 5,000 bases and 100,000 bases. In various embodiments, the two or more sources comprise a maternal chromosome and a paternal chromosome.

In various embodiments, the sequence information from the reference nucleic acids comprises methylation sequence information of the reference nucleic acids. In various embodiments, the methylation sequence information of the target nucleic acids and the methylation sequence information of the reference nucleic acids both comprise methylation statuses for a plurality of genomic sites. In various embodiments, the plurality of genomic sites comprise one or more CpG islands or portions of CpG islands shown in Tables 1-4. In various embodiments, generating sequence information from the target nucleic acids and sequence information from the reference nucleic acids comprises performing an assay, wherein the assay comprises one or more of a. sequencing of target nucleic acids and/or reference nucleic acids via targeted sequencing, whole genome sequencing, or whole genome bisulfite sequencing; b. shallow sequencing and/or deep sequencing; c. a nucleic acid amplification assay; and d. an assay that generates methylation information.

In various embodiments, performing the assay comprises performing both shallow sequencing and deep sequencing. In various embodiments, performing both shallow sequencing and deep sequencing comprises: performing shallow sequencing to generate sequence information from the reference nucleic acids; and performing deep sequencing to generate sequence information from the target nucleic acids. In various embodiments, performing shallow sequencing comprises generating less than less than 50 reads per base, less than 40 reads per base, less than 30 reads per base, less than 20 reads per base, less than 10 reads per base, less than 9 reads per base, less than 8 reads per base, less than 7 reads per base, less than 6 reads per base, or less than 5 reads per base. In various embodiments, performing deep sequencing comprises generating greater than 50 reads per base, greater than 60 reads per base, greater than 70 reads per base, greater than 80 reads per base, greater than 90 reads per base, greater than 100 reads per base, greater than 120 reads per base, greater than 140 reads per base, greater than 150 reads per base, greater than 170 reads per base, greater than 200 reads per base, greater than 225 reads per base, greater than 250 reads per base, greater than 300 reads per base, greater than 400 reads per base, or greater than 500 reads per base.

In various embodiments, the nucleic acid amplification assay is a PCR assay. In various embodiments, the PCR assay comprises a real-time PCR assay, quantitative real-time PCR (qPCR) assay, digital PCR (dPCR) assay, allele-specific PCR assay, or reverse-transcription PCR assay. In various embodiments, generating sequence information from the target nucleic acids and sequence information from the reference nucleic acids comprises performing a target enrichment assay. In various embodiments, the target enrichment assay comprises hybrid capture. In various embodiments, performing the assay comprises: obtaining bisulfite converted target nucleic acids and/or reference nucleic acids; and selectively amplifying target regions of the bisulfite converted target nucleic acids and/or reference nucleic acids. In various embodiments, performing the assay further comprises: determining quantitative values of sequences of the amplicons comprising the amplified target regions to generate the sequence information of the target nucleic acids and/or sequence information of the reference nucleic acids. In various embodiments, the quantitative values comprise cycle threshold (Ct) values. In various embodiments, performing the assay further comprises: sequencing amplicons comprising the amplified target regions to generate the sequence information of the target nucleic acids and/or sequence information of the reference nucleic acids. In various embodiments, the target regions comprise previously identified regions that are differentially methylated in presence of the health condition. In various embodiments, the target regions comprise one or more CpG islands or portions of CpG islands shown in Tables 1-4.

In various embodiments, the sequence information from target nucleic acids is generated from shallow sequencing, and wherein the sequence information from reference nucleic acids is generated from deep sequencing. In various embodiments, shallow sequencing comprises generating less than less than 50 reads per base, less than 40 reads per base, less than 30 reads per base, less than 20 reads per base, less than 10 reads per base, less than 9 reads per base, less than 8 reads per base, less than 7 reads per base, less than 6 reads per base, or less than 5 reads per base. In various embodiments, deep sequencing comprises generating greater than 50 reads per base, greater than 60 reads per base, greater than 70 reads per base, greater than 80 reads per base, greater than 90 reads per base, greater than 100 reads per base, greater than 120 reads per base, greater than 140 reads per base, greater than 150 reads per base, greater than 170 reads per base, greater than 200 reads per base, greater than 225 reads per base, greater than 250 reads per base, greater than 300 reads per base, greater than 400 reads per base, or greater than 500 reads per base.

In various embodiments, the computer program instructions further comprise instructions that, when executed by a processor, cause the processor to: determine a tissue of origin of the health condition using the signal informative of the health condition. In various embodiments, the computer program instructions further comprise instructions that, when executed by a processor, cause the processor to: determine progression of the health condition using the signal informative of the health condition.

In various embodiments, combining the sequence information from the target nucleic acids and the sequence information from the reference nucleic acids comprises determining ratios of methylation levels amongst two or more genomic sites from the target nucleic acids. In various embodiments, the two or more genomic sites are on a common CpG island. In various embodiments, the two or more genomic sites are on different CpG islands. In various embodiments, a subset of the two or more CpG sites are in a common CpG island, and a second subset of the two or more CpG sites are in at least a different CpG island. In various embodiments, combining the sequence information from the target nucleic acids and the sequence information from the reference nucleic acids further comprises determining a difference between the sequence information from target nucleic acids and sequence information from reference nucleic acids to generate a signal that includes limited or no baseline signatures. In various embodiments, combining the sequence information from the target nucleic acids and the sequence information from the reference nucleic acids further comprises determining additional ratios of methylation levels amongst the two or more CpG sites from the signal that includes limited or no baseline signatures. In various embodiments, combining the sequence information from the target nucleic acids and the sequence information from the reference nucleic acids further comprises comparing the ratios of methylation levels amongst two or more CpG sites generated from target nucleic acids and the additional ratios of methylation levels amongst the two or more CpG sites generated from the signal that includes limited or no baseline signatures. In various embodiments, methods disclosed herein further comprise generating a prediction of presence or absence of the health condition based on the comparison. In various embodiments, if the comparison yields no change between the ratios and the additional ratios, then the generated prediction comprises absence of the health condition. In various embodiments, if the comparison yields a change between the ratios and the additional ratios, then the generated prediction comprises presence of the health condition. In various embodiments, the two or more CpG sites are located in CpG islands or portions of CpG islands shown in Tables 1-4.

Additionally disclosed herein is a kit of identifying a cancer signal from an individual, the method comprising: a. equipment to draw one or more samples from an individual, wherein the one or more samples comprise cfDNA and a PBMC DNA; b. a set of detection reagents for determining methylation statuses at a plurality of CpG sites of the cfDNA and the PBMC DNA; and c. instructions for accessing computer program instructions stored on a computer storage medium that, when executed by a processor of a computer system, cause the processor to: compare the methylation status at the plurality of CPG sites of the cfDNA and the PBMC DNA to generate the signal informative of the health condition. In various embodiments, the methylation status was determined from sequencing or nucleic acid amplification. In various embodiments, the nucleic acid amplification comprises a PCR assay. In various embodiments, the PCR assay comprises a real-time PCR assay, quantitative real-time PCR (qPCR) assay, digital PCR (dPCR) assay, allele-specific PCR assay, or reverse-transcription PCR assay. In various embodiments, the CPG sites comprise previously identified CPG sites that are differentially methylated in presence of the health condition. In various embodiments, the CPG sites comprise one or more CpG islands or portions of CpG islands shown in Tables 1-4.

### BRIEF DESCRIPTION OF THE DRAWINGS

These and other features, aspects, and advantages of the present invention will become better understood with regard to the following description and accompanying drawings. It is noted that wherever practicable, similar or like reference numbers may be used in the figures and may indicate similar or like functionality. For example, a letter after a reference numeral, such as “assay apparatus 205A,” indicates that the text refers specifically to the element having that particular reference numeral. A reference numeral in the text without a following letter, such as “assay apparatus 205,” refers to any or all of the elements in the figures bearing that reference numeral (e.g. “assay apparatus 205” in the text refers to reference numerals “assay apparatus 205A,” “assay apparatus 205B,” and/or “assay apparatus 205C” in the figures).

FIG. 1 depicts an overall flow process involving an intra-individual analysis, in accordance with an embodiment.

FIG. 2A depicts an overall system environment including a health condition system, in accordance with an embodiment.

FIG. 2B depicts an example process of combining sequence information of target nucleic acids and reference nucleic acids to generate a signal informative for determining presence or absence of a health condition, in accordance with an embodiment. FIG. 2B discloses SEQ ID NOS 1-3, respectively, in order of appearance.

FIG. 3 shows an example flow process involving an intra-individual analysis, in accordance with an embodiment.

FIG. 4 illustrates an example computer for implementing the entities shown in FIGS. 1, 2A, 2B, and 3.

FIG. 5 shows an example sample from which target nucleic acids and reference nucleic acids are obtained.

### DETAILED DESCRIPTION

### Definitions

Terms used in the claims and specification are defined as set forth below unless otherwise specified.

The terms “subject,” “patient,” and “individual” are used interchangeably and encompass a cell, tissue, or organism, human or non-human, male or female.

The term “sample” can include a single cell or multiple cells or fragments of cells or an aliquot of body fluid, such as a blood sample, taken from a subject, by means including venipuncture, excretion, ejaculation, massage, biopsy, needle aspirate, lavage sample, scraping, surgical incision, or intervention or other means known in the art. Examples of an aliquot of body fluid include amniotic fluid, aqueous humor, bile, lymph, breast milk, interstitial fluid, blood, blood plasma, cerumen (earwax), Cowper's fluid (pre-ejaculatory fluid), chyle, chyme, female ejaculate, menses, mucus, saliva, urine, vomit, tears, vaginal lubrication, sweat, serum, semen, sebum, pus, pleural fluid, cerebrospinal fluid, synovial fluid, intracellular fluid, and vitreous humour. In particular embodiments, the sample is a liquid biopsy sample, such as a blood sample.

The term “obtaining sequence information” encompasses obtaining information that is determined from at least one sample. Obtaining sequence information encompasses obtaining a sample and processing the sample and/or performing an assay on the sample to experimentally determine the sequence information. The phrase also encompasses receiving the information, e.g., from a third party that has processed the sample and/or performed an assay on the sample to experimentally determine the sequence information.

The phrase “target nucleic acids” refers to nucleic acids of an individual that contain at least signatures that may be informative for determining presence or absence of the health condition. The target nucleic acids may further include baseline biological signatures of the individual that are not informative or less informative. In various embodiments, target nucleic acids may be nucleic acids derived from a diseased cell that is associated with the health condition. For example, target nucleic acids may be cell-free nucleic acids originating from cancer cells. Target nucleic acids can be any of DNA, cDNA, or RNA. In particular embodiments, target nucleic acids include DNA. In various embodiments, target nucleic acids may be cell-free nucleic acids originating from cancer cells that then undergo deep sequencing. Thus, reads from such target nucleic acids that are generated via deep sequencing can contain both baseline biological signatures and signatures that may be informative for determining presence or absence of the health condition.

The phrase “reference nucleic acids” refers to nucleic acids of an individual that contain baseline biological signatures of the individual. Here, the baseline biological signatures of the individual may be present when the individual is healthy, and therefore, the baseline biological signatures are less informative for determining presence or absence of the health condition in comparison to sequence information of the target nucleic acids. Reference nucleic acids can be any of DNA, cDNA, or RNA. In particular embodiments, reference nucleic acids include DNA. In some embodiments, reference nucleic acids are obtained from non-cancerous cells, e.g., peripheral blood mononuclear cells (PBMCs) or polymorphonuclear cells. In other embodiments, reference nucleic acids may be obtained from cell-free nucleic acids (e.g., from a liquid biopsy) that then undergo shallow sequencing. These cell-free nucleic acids may be a mixture of nucleic acids from cancerous and non-cancerous cells. Thus, reads from such reference nucleic acids that are generated via shallow sequencing can contain baseline biological signatures. These reads can further lack or have minimal signatures that may be informative for determining presence or absence of the health condition.

It must be noted that, as used in the specification, the singular forms “a,” “an” and “the” include plural referents unless the context clearly dictates otherwise.

Overview

Disclosed herein are methods for performing an intra-individual analysis to determine a presence or absence of one or more health conditions within a patient. For a particular patient, the intra-individual analysis is performed to remove baseline biological signatures that are present in the patient irrespective of whether the patient has a health condition or does not have the health condition. Thus, these baseline biological signatures would be confounding signals if analyzed to predict whether the patient has a presence or absence of the health condition. Performing the intra-individual analysis eliminates these confounding baseline biological signatures while keeping signatures that are more informative for determining presence or absence of the health condition. For example, in processing nucleic acid sequencing information to generate a signal that may be detected, the resulting signal may comprise a mixture of baseline biological signatures (e.g., germline methylation in a patient) that represent a form of background noise and signatures informative of a health condition (e.g., cancer). Such background noise can obscure a signal informative of a health condition. Advantageously, in certain embodiments, methods described herein contemplate subtracting such background noise from a patient's nucleic acid sequencing information, thereby improving the signal-to-noise ratio of the signal informative of a health condition.

In contrast to an inter-individual analysis, where, for example, to determine a presence or absence of one or more health conditions within a patient, an average of baseline signatures from a group of normal subjects are removed from the nucleic acid sequencing information of the patient, it has been discovered that performing an intra-individual analysis can significantly improve the sensitivity or specificity of detecting a signal informative for determining presence or absence of the health condition.

Generally, the intra-individual analysis involves generating information from at least target nucleic acids and reference nucleic acids from one or more samples obtained from the patient. In various embodiments, the generated information includes sequence information of the target nucleic acids and sequence information of the reference nucleic acids. The intra-individual analysis involves combining the information from the target nucleic acids and the reference nucleic acids to generate a signal informative for determining presence or absence of one or more health conditions within the patient. By combining the information from the target nucleic acids and the reference nucleic acids, the generated signal can be more informative of presence or absence of a health condition in comparison to a signal derived from the target nucleic acids alone. For example, the information from the reference nucleic acids can represent baseline biology of the patient. By combining the information from the target nucleic acids and the reference nucleic acids, the baseline biology of the patient, which may not be informative for the presence or absence of a health condition, is removed from the generated signal. Thus, information of the target nucleic acids that are not attributable to the patient's baseline biology remains and is included in the generated signal for determining presence or absence of one or more health conditions in the patient.

In various embodiments, the intra-individual analysis can be performed for predicting presence or absence of two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, or twenty or more different health conditions. In particular embodiments, the health conditions are forms of cancer. In particular embodiments, the intra-individual analysis can be performed for predicting presence or absence of one of ten or more different cancers. In particular embodiments, the intra-individual analysis can be performed for predicting presence or absence of one of fifteen or more different cancers. In particular embodiments, the intra-individual analysis can be performed for predicting presence or absence of one of twenty or more different cancers. In particular embodiments, the different cancers are early stage cancers or preclinical stage cancers. Further examples of health conditions are detailed herein.

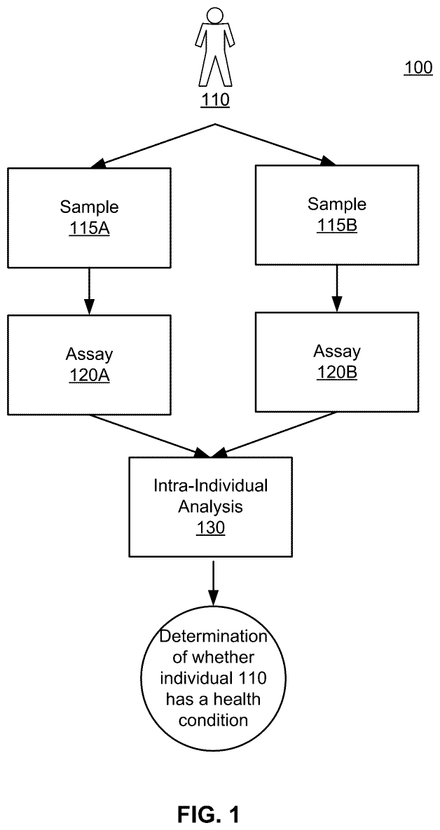

FIG. 1 depicts an overall flow process 100 involving an intra-individual analysis, in accordance with an embodiment. Although FIG. 1 shows the flow process in relation to a single individual 110, in various embodiments, the flow process 100 can be performed for more than a single individual 110 (e.g., for thousands, millions, tens of millions, or hundreds of millions of individuals).

As shown in FIG. 1, one or more samples 115 (e.g., sample 115A and/or sample 115B) are obtained from the individual 110. In particular embodiments, the one or more samples 115 obtained from the individual 110 are blood samples. The samples 115 can be obtained by the individual or by a third party, e.g., a medical professional. Examples of medical professionals include physicians, emergency medical technicians, nurses, first responders, psychologists, phlebotomist, medical physics personnel, nurse practitioners, surgeons, dentists, and any other obvious medical professional as would be known to one skilled in the art. In various embodiments, the one or more samples 115 can be obtained from the individual 110 by a reference lab.

In various embodiments, the sample obtained from the individual is a liquid biopsy sample. In various embodiments, the liquid biopsy sample may include various biomarkers, examples of which include proteins, metabolites, and/or nucleic acids. In particular embodiments, the liquid biopsy sample includes cell-free DNA (cfDNA) fragments. In particular embodiments, the liquid biopsy sample includes one or more cells in the sample, wherein the one or more cells include nucleic acids, such as genomic DNA.

As shown in the embodiment in FIG. 1, a sample 115A and a sample 115B can be obtained from the individual 110. In various embodiments, one of the samples contains target nucleic acids and the other of the samples contains reference nucleic acids. Therefore, in such embodiments, target nucleic acids can be obtained from one of the samples, and reference nucleic acids can be obtained from the other of the samples. Separate assays (e.g., assay 120A and assay 120B) can be performed on the target nucleic acids and the reference nucleic acids.

In various embodiments, target nucleic acids and reference nucleic acids can be obtained from a single sample. For example, instead of different samples 115A and 115B, a single sample 115 may be obtained from the individual 110. Target nucleic acids and reference nucleic acids are separately obtained from the single sample. In various embodiments, the sample is processed to separate the target nucleic acids and reference nucleic acids. For example, the sample be processed through any one of centrifugation, filtration, gel electrophoresis, bead capture, or matrix extraction. In particular embodiments, target nucleic acids are cell-free nucleic acids and therefore, can be obtained from the supernatant of the separated sample. In particular embodiments, reference nucleic acids are cellular genomic nucleic acids and therefore, can be obtained from a different portion of the separated sample that contains cells.

In particular embodiments, a sample 115 obtained from the individual is a blood sample that contains target nucleic acids as well as reference nucleic acids. Target nucleic acids may include signatures that are informative of determining presence or absence of a health condition, and can further include baseline biological signatures. Here, target nucleic acids in the blood sample may be derived from a diseased cell which is associated with the health condition. For example, target nucleic acids can include cell-free DNA in the blood that originates from a diseased cell. In particular embodiments, target nucleic acids are cell-free DNA in the blood that originates from a cancer cell.

Reference nucleic acids in the sample refer to nucleic acids that contain baseline biological signatures of the individual. For example, baseline biological signatures of the individual may be present in nucleic acids irrespective of whether the nucleic acids originate from a diseased source, or a non-diseased source. The baseline biological signatures of the reference nucleic acids are generally less informative for determining presence or absence of a health condition in comparison to the informative signatures present in the target nucleic acids. In various embodiments, reference nucleic acids refer to cellular genomic DNA derived from a healthy cell from the individual. In various embodiments, reference nucleic acids found in the sample derive from a cell in a healthy organ of the individual. Example organs include the brain, heart, thorax, lung, abdomen, colon, cervix, pancreas, kidney, liver, muscle, lymph nodes, esophagus, intestine, spleen, stomach, and gall bladder. In particular embodiments, reference nucleic acids are found in the sample and refer to cellular genomic DNA or germline DNA derived from a non-cancerous cells, e.g., peripheral blood mononuclear cells (PBMCs) or polymorphonuclear cells.

In various embodiments, a plurality of samples 115 are obtained from the individual 110 at a plurality of different points in time. For example, a first sample 115A can be obtained at a first timepoint and at least a second sample 115B can be obtained from the individual 110 at a second timepoint. Obtaining a plurality of samples 115 from the individual at a plurality of different points in time includes obtaining a number M of samples 115, wherein M is one of: 2, 3, 4, . . . , N−1, N, wherein N is a positive integer. In such embodiments, target nucleic acids and reference nucleic acids can be obtained at the different points in time, thereby enabling intra-individual analyses across the different points in time. This can enable the tracking of progression of a health condition over the different points in time.

In various embodiments, samples (e.g., sample 115A and/or sample 115B) may be processed to extract the target nucleic acids and reference nucleic acids. In various embodiments, samples can undergo cellular disruption methods (e.g., to obtain genomic DNA) involving chemical methods or mechanical methods. Example chemical methods include osmotic shock, enzymatic digestion, detergents, or alkali treatment. Example mechanical methods include homogenization, ultrasonication or cavitation, pressure cell, or ball mill. In various embodiments, samples can undergo removal of membrane lipids or proteins or nucleic acid purification. Example chemical methods for removing membrane lipids or proteins and methods for nucleic acid purification include guanidine thiocyanate (GuSCN)-phenol-chloroform extraction, alkaline extraction, cesium chloride gradient centrifugation with ethidium bromide, Chelex® extraction, or cetyltrimethylammonium bromide extraction. Example physical methods for removing membrane lipids or proteins and methods for nucleic acid purification include solid-phase extraction methods using any of silica matrices, glass particles, diatomaceous earth, magnetic beads, anion exchange material, or cellulose matrix. Further details of nucleic acid extraction methods are described in Ali et al, Current Nucleic Acid Extraction Methods and Their Implications to Point-of-Care Diagnostics, Biomed Res. Int. 2017; 2017:9306564, which is hereby incorporated by reference in its entirety.

One or more assays (e.g., assay 120A and/or assay 120B) are performed on the obtained sample 115A and/or sample 115B to generate sequence information. Generally, assays are performed to generate sequence information for target nucleic acids and to generate sequence information for reference nucleic acids. In particular embodiments, sequence information includes statuses for a plurality of genomic sites, such as epigenetic statuses for a plurality of CpG sites. In various embodiments, epigenetic statuses refer to methylation statuses. In particular embodiments, sequence information of the target nucleic acids and sequence information of the reference nucleic includes statuses for two or more, three or more, four or more, five or more, six or more, seven or more, eight or more, nine or more, or ten or more common genomic sites. In particular embodiments, sequence information of the target nucleic acids and sequence information of the reference nucleic each includes statuses for 15 or more, 20 or more, 25 or more, 30 or more, 40 or more, 50 or more, 100 or more, 200 or more, 300 or more, 400 or more, 500 or more, 750 or more, 1000 or more, 2000 or more, 3000 or more, 4000 or more, 5000 or more, 6000 or more, 7000 or more, 8000 or more, 9000 or more, 10000 or more, 11000 or more, 12000 or more, 13000 or more, 14000 or more, 15000 or more, 16000 or more, 17000 or more, 18000 or more, 19000 or more, or 20000 or more genomic sites. In particular embodiments, sequence information of the target nucleic acids and sequence information of the reference nucleic each includes statuses for 15 or more, 20 or more, 25 or more, 30 or more, 40 or more, 50 or more, 100 or more, 200 or more, 300 or more, 400 or more, 500 or more, 750 or more, 1000 or more, 2000 or more, 3000 or more, 4000 or more, 5000 or more, 6000 or more, 7000 or more, 8000 or more, 9000 or more, 10000 or more, 11000 or more, 12000 or more, 13000 or more, 14000 or more, 15000 or more, 16000 or more, 17000 or more, 18000 or more, 19000 or more, or 20000 or more of the same genomic sites or overlapping genomic sites. In various embodiments, the plurality of genomic sites include a plurality of CpG islands (CGIs) whose differential methylation status may be indicative of a health condition. Further details regarding the assay 120A or assay 120B are described herein.

Although FIG. 1 shows two separate assays (e.g., assay 120A and assay 120B) performed on two separate samples (e.g., sample 115A and sample 115B), in various embodiments, more or fewer assays can be performed or more or fewer samples. In particular embodiments, a single sample 115 is obtained from the individual. In some embodiments, two assays (e.g., assay 120A and assay 120B) are performed on the single sample to generate sequence information for target nucleic acids and sequence information for reference nucleic acids. In some embodiments, a single assay is performed on the single sample to generate sequence information for target nucleic acids and sequence information for reference nucleic acids.

The intra-individual analysis 130 involves combining the sequence information of the target nucleic acids and the sequence information of the reference nucleic acids to generate a signal informative for determining presence or absence of a health condition. Here, the signal informative for determining presence or absence of a health condition is more informative for determining presence or absence of the health condition in comparison to the sequence information of the target nucleic acids alone. In particular embodiments, the signal informative for determining presence or absence of the health condition includes informative signatures from the target nucleic acids (e.g., signatures derived from diseased cells) and excludes baseline biological signatures (e.g., baseline biological signatures present in reference nucleic acids). Further details of the intra-individual analysis 130, and specifically the generation of the signal informative for determining presence or absence of the health condition, is described herein.

In various embodiments, the intra-individual analysis 130 involves analyzing the signal to predict whether the individual has the health condition. Thus, as shown in FIG. 1, the output of the intra-individual analysis 130 can be a determination of whether the individual has the health condition. In various embodiments, the determination can be useful for guiding the decision-making for treating the individual. For example, if the determination reveals that the individual has the health condition, the individual can be provided a therapy (e.g., a prophylactic therapy or a preventative therapy) to treat the health condition.

Health Condition System

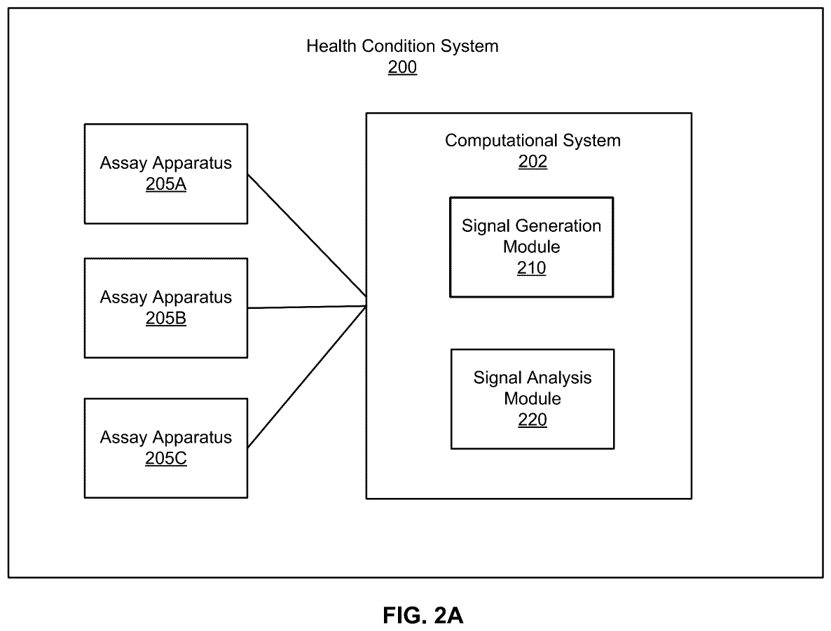

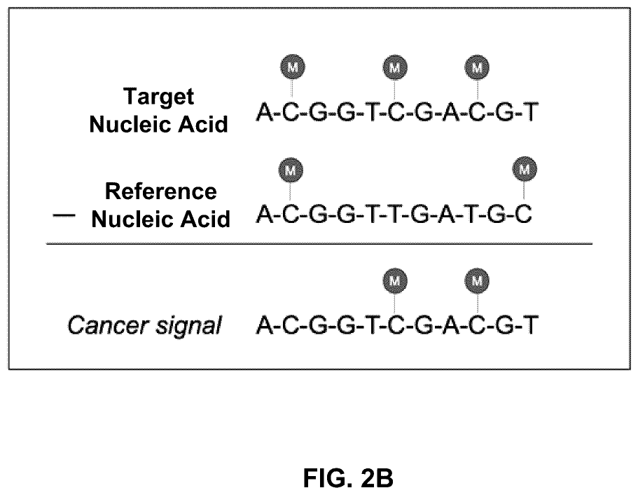

FIG. 2A depicts an overall system environment including a health condition system, in accordance with an embodiment. The block diagram of the health condition system 200 is introduced to show an embodiment in which the health condition system 200 includes one or more assay apparatus 205 communicatively coupled to a computational system 202. The computational system 202 can further include computational modules, such as a signal generation module 210 and a signal analysis module 220. FIG. 2A depicts an embodiment in which the health condition system 200 performs one or more assays (e.g., assay 120A or 120B described in FIG. 1) and performs the intra-individual analysis (e.g., intra-individual analysis 130 described in FIG. 1).

In various embodiments, the health condition system 200 may be differently configured than shown in FIG. 2A. For example, although the health condition system 200 shown in FIG. 2A includes three different assay apparatus 205, in various embodiments, the health condition system 200 includes fewer or additional assay apparatus. In particular embodiments, the health condition system 200 does not include an assay apparatus. In such embodiments, the health condition system 200 includes only the computational system 202. In these embodiments in which the health condition system 200 does not include an assay apparatus, the health condition system 200 may perform the intra-individual analysis (e.g., intra-individual analysis 130 shown in FIG. 1). However, the health condition system 200 does not obtain samples or perform assays. The assay apparatus 205 may be operated and used by a different entity, such as a third party entity. Thus, the third party entity can perform assays using one or more assay apparatus 205 and then transmits the data generated from the assays to the health condition system 200 for performing the intra-individual analysis.

Referring to FIG. 2A, the signal generation module 210 combines sequence information from target nucleic acids and sequence information from reference nucleic acids to generate a signal informative for determining presence or absence of a health condition in an individual. Further details of steps performed by the signal generation module 210 are described herein.

The signal analysis module 220 analyzes the signal informative for determining the presence or absence of the health condition and generates a prediction as to whether the health condition is present in the individual. Further details of steps performed by the signal analysis module 220 are described herein.

Assays

Methods disclosed herein involve performing an assay to generate sequence information for target nucleic acids and/or reference nucleic acids. Assays described in this section can refer to either assay 120A, assay 120B, or both assay 120A and assay 120B shown in FIG. 1. Referring to FIG. 2A, performing an assay can involve employing one or more assay apparatus 205 to perform the assay.

In various embodiments, sequence information of target nucleic acids and/or sequence information of reference nucleic acids refer to statuses for a plurality of genomic sites. Sequence information of target nucleic acids refers to epigenetic statuses (e.g., methylation statuses) across a plurality of genomic sites in the target nucleic acids. Sequence information of reference nucleic acids refers to epigenetic statuses (e.g., methylation statuses) across a plurality of genomic sites in the reference nucleic acids. In various embodiments, the plurality of genomic sites are previously identified and selected. For example, the plurality of genomic sites may be one or more CpG sites whose differential methylation are informative for determining whether an individual has a health condition. A CpG site is portion of a genome that has cytosine and guanine separated by only one phosphate group and is often denoted as “5′-C-phosphate G-3′”, or “CpG” for short. Regions with a high frequency of CpG sites are commonly referred to as “CG islands” or “CGIs”. It has been found that certain CGIs and certain features of certain CGIs in tumor cells tend to be different from the same CGIs or features of the CGIs in healthy cells. Herein, such CGIs and features of the genome are referred to herein as “cancer informative CGIs.” Cancer informative CGI can be a “CGI identifier” or reference number to allow referencing CGIs during data processing by their respective unique CGI identifiers. Example CGIs include, but are not limited to, the CGIs shown in the accompanying tables (referred to herein as Tables 1-4) which lists, for each CGI, its respective location in the human genome. Additional example CGIs are disclosed in WO2018209361 (see Table 1) and WO2022133315 (see Table 2 entitled “TOO Methylation Sites” and Table 3 entitled “Pan Cancer Methylation Sites”), each of which is hereby incorporated by reference in its entirety. In some embodiments, methylation statuses of a plurality of CpGs within a CGI may be analyzed. In some embodiments, at least a portion of the CpGs within a CGI may be analyzed. In other embodiments, all of the CpGs within a CGI may be analyzed. In some embodiments, an analysis of a CGI as contemplated herein may comprise analyzing CpGs within at least a portion of one or more regions in Tables 1-4.

In various embodiments, performing an assay to generate sequence information for a plurality of genomic sites includes the steps of processing nucleic acids of a sample, enriching the processed nucleic acids for pre-selected genomic sequences (e.g., pre-selected informative CGIs), amplifying the genomic sequences to generate amplicons, and quantifying the amplicons including the genomic sequences (e.g., via sequencing such as next generation sequencing or via quantitative methods such as an ELISA, quantitative PCR, allele-specific PCR, or DNA or RNA-based assay). In various embodiments, performing an assay to generate sequence information for a plurality of genomic sites involves a subset of the previously mentioned steps. For example, enriching the processed nucleic acids can be omitted. Therefore, performing an assay may include processing nucleic acids of a sample, amplifying the pre-selected genomic sequences, and quantifying the amplicons including the genomic sequences.

In various embodiments, performing an assay (e.g., assay 120A or assay 120B) involves processing nucleic acids (e.g., cfDNA fragments) from a sample (e.g., liquid biopsy sample). In various embodiments, processing nucleic acids includes treating the nucleic acids to capture methylation modifications. In various embodiments, processing nucleic acids to capture methylation modifications includes performing deamination of cytosine residues. Other techniques include but are not limited to enzymatic methods. In various embodiments, processing nucleic acids to capture methylation modifications includes performing any of nucleic acid amplification, polymerase chain reaction (PCR), methylation specific PCR, bisulfite pyrosequencing, single-strand conformation polymorphism (SSCP) analysis, methylation-sensitive single-strand conformation analysis restriction analysis, high resolution melting analysis, methylation-sensitive single-nucleotide primer extension, restriction analysis, microarray technology, next generation methylation sequencing, nanopore sequencing, and combinations thereof.

In various embodiments, performing deamination of cytosine residues is useful for determining methylation statuses of nucleic acids from a sample. Performing deamination involves providing or exposing nucleic acids from a sample to a deaminating agent. In various embodiments, performing deamination of cytosine residues involves performing selective deamination. Selective deamination refers to a process in which cytosine residues are selectively deaminated over 5-methylcytosine residues. Deamination of cytosine forms uracil, effectively inducing a C to T point mutation to allow for detection of methylated cytosines. Methods of deaminating cytosine are known in the art, and include bisulfite conversion and enzymatic conversion. Bisulfite conversion enables highly efficient conversion of unmethylated cytosines to uracils of DNA from samples such as whole blood or plasma, cultured cells, tissue samples, genomic DNA, and formalin-fixed, paraffin-embedded (FFPE) tissues. Bisulfite conversion can be performed using commercially available technologies, such as Zymo Gold available from Zymo Research (Irvine, CA) or EpiTect Fast available from Qiagen (Germantown, MD). In certain embodiments, the enzymatic conversion comprises subjecting the nucleic acid to TET2, which oxidizes methylated cytosines, thereby protecting them, and subsequent exposure to APOBEC, which converts unprotected (unmethylated) cytosines to uracils.

In various embodiments, performing the assay includes enriching for specific sequences in the target nucleic acids and/or reference nucleic acids. In various embodiments, the specific sequences refer to sequences of pre-selected CGIs. In various embodiments, enrichment of pre-selected CGIs can be accomplished via hybrid capture. Examples of such hybrid capture probe sets include the KAPA HyperPrep Kit and SeqCAP Epi Enrichment System from Roche Diagnostics (Pleasanton, CA). For example, hybrid capture probe sets can be designed to hybridize with particular sequences of the target nucleic acids and/or reference nucleic acids, thereby capturing and enriching the particular sequences.

In various embodiments, performing the assay includes performing nucleic acid amplification to amplify the particular sequences of the target nucleic acids and/or reference nucleic acids. Examples of such assays include, but are not limited to performing PCR assays, Real-time PCR assays, Quantitative real-time PCR (qPCR) assays, digital PCR (dPCR), Allele-specific PCR assays, Reverse-transcription PCR assays and reporter assays. For example, given the processed nucleic acids (e.g., bisulfite converted nucleic acids) that are enriched for pre-selected sequences, a PCR assay is performed to amplify the pre-selected sequences to generate amplicons. Here, PCR primers are added to initiate the amplification. In various embodiments, the PCR primers are whole genome primers that enable whole genome amplification. In various embodiments, the PCR primers are gene-specific primers that result in amplification of sequences of specific genes. In various embodiments, the PCR primers are allele-specific primers. For example, allele specific primers can target a genomic sequence corresponding to a pre-selected CGI, such that performing nucleic acid amplification results in amplification of the sequence of the pre-selected CGI.

In various embodiments, performing the assay includes quantifying the nucleic acids including the pre-selected sequences (e.g., informative CGIs). In some embodiments, quantifying the nucleic acids to generate sequence information comprises performing any of real-time PCR assay, quantitative real-time PCR (qPCR) assay, digital PCR (dPCR) assay, allele-specific PCR assay, or reverse-transcription PCR assay. Therefore, the number of methylated, hypermethylated, unmethylated, or partially methylated pre-selected sequences are quantified.

In various embodiments, performing the assay comprises sequencing the nucleic acids including the pre-selected sequences. Thus, the sequenced reads are aligned to a reference library and sequence information including methylation statuses of the informative CGIs of amplicons derived from the target nucleic acids and/or reference nucleic acids can be determined. Therefore, the number of methylated, hypermethylated, unmethylated, or partially methylated pre-selected sequences of the target nucleic acids and the reference nucleic acids can be quantified via the sequenced reads.

In various embodiments, performing the assay comprises performing at least two different types of sequencing, such as sequencings of different depth. For example, a first type of sequencing can include shallow sequencing and a second type of sequencing can include deep sequencing. Generally, shallow sequencing and deep sequencing differ in the number of sequence reads that are generated (e.g., generated for a cell or generated for a target region). Deep sequencing can involve sequencing particular target regions multiple times, such as hundreds or thousands of times to generate a large number of reads, whereas shallow sequencing can involve generating fewer reads, often with the goal of achieving higher coverage across the genome. Example assays for shallow sequencing include shallow shotgun sequencing or shallow whole genome sequencing (e.g., using Ion ReproSeq PGS Kit from Thermo Fisher Scientific).

In various embodiments, shallow sequencing may generate M number of reads per base (e.g., M average number of reads per base), whereas deep sequencing may generate N number of reads per base (e.g., N average number of reads per base), where Nis significantly larger than M. In various embodiments, M is less than 100 reads per base, less than 90 reads per base, less than 80 reads per base, less than 70 reads per base, less than 60 reads per base, less than 50 reads per base, less than 40 reads per base, less than 30 reads per base, less than 20 reads per base, less than 10 reads per base, less than 9 reads per base, less than 8 reads per base, less than 7 reads per base, less than 6 reads per base, or less than 5 reads per base. In various embodiments, Nis greater than 10 reads per base, greater than 20 reads per base, greater than 25 reads per base, greater than 30 reads per base, greater than 40 reads per base, greater than 50 reads per base, greater than 60 reads per base, greater than 70 reads per base, greater than 80 reads per base, greater than 90 reads per base, greater than 100 reads per base, greater than 120 reads per base, greater than 140 reads per base, greater than 150 reads per base, greater than 170 reads per base, greater than 200 reads per base, greater than 225 reads per base, greater than 250 reads per base, greater than 300 reads per base, greater than 400 reads per base, or greater than 500 reads per base.

In various embodiments, shallow sequencing may generate W number of reads per cell, whereas deep sequencing may generate X number of reads per cell, where X is significantly larger than W. In various embodiments, W is less than 200,000 reads per cell, less than 100,000 reads per cell, less than 50,000 reads per cell, less than 40,000 reads per cell, less than 30,000 reads per cell, less than 20,000 reads per cell, or less than 10,000 reads per cell. In various embodiments, X is greater than 200,000 reads per cell, greater than 300,000 reads per cell, greater than 400,000 reads per cell, greater than 500,000 reads per cell, greater than 600,000 reads per cell, greater than 700,000 reads per cell, greater than 800,000 reads per cell, greater than 900,000 reads per cell, or greater than 1 million reads per cell.

In various embodiments, shallow sequencing may generate Y number of reads for a particular target region (e.g., a target region including one or more CpG islands or portions of CpG islands shown in Tables 1-4), whereas deep sequencing may generate Z number of reads for a particular target region (e.g., a target region including one or more CpG islands or portions of CpG islands shown in Tables 1-4), where Z is significantly larger than Y. In various embodiments, Y is less than 1000 reads for the target region, less than 500 reads for the target region, less than 400 reads for the target region, less than 300 reads for the target region, less than 200 reads for the target region, less than 100 reads for the target region, less than 50 reads for the target region, or less than 30 reads for the target region. In various embodiments, Z is greater than 100 reads for the target region, greater than 200 reads for the target region, greater than 300 reads for the target region, greater than 400 reads for the target region, greater than 500 reads for the target region, greater than 600 reads for the target region, greater than 700 reads for the target region, greater than 800 reads for the target region, greater than 900 reads for the target region, greater than 1000 reads for the target region, greater than 2500 reads for the target region, greater than 5000 reads for the target region, greater than 10,000 reads for the target region, greater than 20,000 reads for the target region, greater than 30,000 reads for the target region, greater than 40,000 reads for the target region, greater than 50,000 reads for the target region, or greater than 100,000 reads for the target region.

In various embodiments, performing the assay comprises sequencing the target nucleic acids and/or reference nucleic acids. In various embodiments, sequencing comprises performing next generation sequencing methods to generate sequence reads from the target nucleic acids and/or reference nucleic acids (e.g., sequence reads that include one or more CpG islands or portions of CpG islands shown in Tables 1-4). As described herein, sequence reads of reference nucleic acids may be long sequence reads (e.g., greater than 500 bases in length). Generally, long sequence reads include an average read length that is longer than sequence reads obtained through standard sequencing methods. In various embodiments, the long sequence reads from reference nucleic acids refer to sequence reads of at least 500 bases, at least 1 kilobase, at least 2 kilobases (kb), at least 3 kb, at least 4 kb, at least 5 kb, at least 6 kb, at least 7 kb, at least 8 kb, at least 9 kb, at least 10 kb, at least 12 kb, at least 15 kb, at least 20 kb, at least 25 kb, at least 30 kb, at least 40 kb, at least 50 kb, at least 60 kb, at least 70 kb, at least 80 kb, at least 90 kb, at least 100 kb, at least 200 kb, at least 300 kb, at least 400 kb, at least 500 kb, at least 600 kb, at least 700 kb, at least 800 kb, at least 900 kb, at least 1000 kb, at least 1500 kb, or at least 2000 kb. In particular embodiments, the long sequence reads of reference nucleic acids refer to sequence reads of between 5 kb and 100 kb, between 10 kb and 80 kb, between 20 kb and 70 kb, between 30 kb and 60 kb, or between 40 kb and 50 kb. In particular embodiments, long sequence reads of reference nucleic acids refer to sequence reads of greater than about 8 kb, greater than about 9 kb or greater than about 10 kb. In particular embodiments, long sequence reads of reference nucleic acids refer to sequence reads between about 10 kb and about 100 kb, or between about 10 kb and about 2 MB. In various embodiments, generating long sequence reads of reference nucleic acids involves performing nanopore sequencing. Methods for long-read sequencing are known in the art and such methods can be performed using, for example, an Oxford Nanopore instrument (e.g., PromethION™) or Pacific Biosciences Single-Molecule Real-Time (SMRT) sequencing technology.

In various embodiments, performing the assay includes generating phased sequencing information for target nucleic acids and/or reference nucleic acids. As used herein, “phased sequencing information,” also referred to herein as “haplotype sequencing information,” refers to sequencing information derived specifically from a particular source. For example, phased sequencing information or haplotype sequencing information can refer to sequencing information derived from either the maternal or paternal chromosome. Generally, phased sequencing information of target nucleic acids may be useful for determining presence or absence of a cancer because signals originating from the same source (e.g., maternal or paternal chromosome) may provide additional information in comparison to other approaches that merely analyze signals irrespective of the source.

In various embodiments, the phased sequencing information comprises mutation sequence information of the cell-free DNA. For example, mutation sequence information can include one or more mutations present across a plurality of genomic sites. In particular embodiments, the mutation sequence information includes one or more mutations that originate from a common source (e.g., a maternal chromosome or a paternal chromosome). Here, two or more genomic sites derived from a common source with a particular pattern (e.g., that each have mutations, or one site has a mutation and the second site does not have a mutation, or neither site has a mutation) can be referred to as coupled genomic sites. In various embodiments, a mutation can be any of a single nucleotide polymorphism (SNP), single nucleotide variant (SNV), insertion, deletion, copy number variation (CNV), duplication, or translocation.

In various embodiments, the phased sequencing information comprises methylation sequence information of the cell-free DNA. Methylation sequence information can include methylation statuses across a plurality of genomic sites. In particular embodiments, the methylation sequence information includes methylation statuses of genomic sites from a common source (e.g., a maternal chromosome or a paternal chromosome). As a specific example, methylation status at a first genomic site may be coupled with methylation status at a second genomic site on the same maternal or paternal chromosome. Two or more genomic sites with a particular methylation pattern (e.g., all methylated, partially methylated, or non-methylated) that originate from the same maternal or paternal chromosome is referred to herein as coupled methylation sites. Example coupled methylation sites may be two or more CGIs disclosed herein (e.g., two or more CGIs or portions of CpG islands shown in Tables 1-4). In various embodiments, two or more genomic sites of coupled methylation sites may be separated by tens, hundreds, or even thousands of bases. Thus, coupled methylation sites include two or more genomic sites from a common source and need not be limited to genomic sites that are close in proximity (e.g., adjacent CpG sites). In various embodiments, coupled methylation sites include 3 or more, 4 or more, 5 or more, 6 or more, 7 or more, 8 or more, 9 or more, 10 or more, 15 or more, 20 or more, 25 or more, 30 or more, 35 or more, 40 or more, 45 or more, 50 or more, 60 or more, 70 or more, 80 or more, 90 or more, 100 or more, 200 or more, 300 or more, 400 or more, 500 or more, 600 or more, 700 or more, 800 or more, 900 or more, or 1000 or more sites from a common source. Thus, detecting these coupled methylation sites may provide disease detection utility.

In various embodiments, generating phased sequencing information for target nucleic acids comprises aligning sequence reads of target nucleic acids to long sequence reads of reference nucleic acids derived from different sources (e.g., either the maternal or paternal chromosomes). Different long sequence reads of reference nucleic acids originating from different sources can be distinguished due to sequence differences present in the long sequence reads. For example, given a particular chromosome, long sequence reads derived from a maternal chromosome would have sequence differences in comparison to long sequence reads derived from a paternal chromosome. Here, sequence differences can refer to mutations that are present in long sequence reads from one source, but not present in long sequence reads from the second source, and vice versa. Thus, the presence or absence of certain mutations can be useful for distinguishing whether a long sequence read originated from a first source or a second source. Altogether, by comparing sequences of long sequence reads, a first set of long sequence reads with a set of common sequences can be attributed to a first source (e.g., a maternal chromosome) whereas a second set of long sequence reads with a different set of common sequences can be attributed to a second source (e.g., a paternal chromosome). In various embodiments, the different sets of long sequence reads need not specifically be attributed to a maternal chromosome and a paternal chromosome; rather, it is sufficient to distinguish different sets of long sequence reads from a first source and a second source. These long sequence reads from a first source or a second source have sufficiently different sequences to enable phasing of the target nucleic acids (e.g., to determine the sources from which the target nucleic acids were derived).

By aligning sequence reads of target nucleic acids to long sequence reads of reference nucleic acids, the long sequence reads of reference nucleic acids serve as digital guides to phase e.g., they determine the source of target nucleic acids. For example, target nucleic acids from a first common source (e.g., from a maternal chromosome) can be categorized together based on sequence similarities between the target nucleic acids and the long sequence reads of reference nucleic acids from the first source. Additionally, target nucleic acids from a second common source (e.g., from a paternal chromosome) can be categorized together based on sequence similarities between the target nucleic acids and the long sequence reads of reference nucleic acids from the second source. In contrast to using the standard human genome to align sequence reads of target nucleic acids, using long reads of reference nucleic acids would enable alignment of reference nucleic acids to sequences of the maternal or paternal chromosome Individual-specific differences between target nucleic acids deriving from the maternal and paternal chromosomes could be used as markers to create haplotype-specific sequence information that is informative for determining presence or absence of a cancer.

In various embodiments, phased sequencing information includes phased methylation sequencing information of cfDNA, where at least a first set of the phased methylation sequencing information of cfDNA originates from a first source and at least a second set of the phased methylation sequencing information of cfDNA originates from a second source. In various embodiments, methods for generating phased sequencing information can further include comparing the first set of the phased methylation sequencing information from cfDNA from the first source to the second set of the phased methylation sequencing information from cfDNA from the second source. In particular embodiments, generating phased sequencing information further includes comparing methylation statuses of two or more genomic sites from a first source to methylation statuses of the same two or more genomic sites from a second source. Differences in methylation statuses of genomic sites from the first source and the second source can be included in the signal informative for determining presence or absence of a cancer. For example if multiple genomic sites from a first source (e.g., maternal chromosome) are methylated but the same genomic sites from a second source (e.g., paternal chromosome) are unmethylated, the differential methylation of the genomic sites may be an informative signal for presence or absence of a cancer.

Intra-Individual Analysis

The description in this section pertains to the performance of an intra-individual analysis, such as an intra-individual analysis 130 described in FIG. 1, which can be performed by the health condition system 200 described in FIG. 2A. Generally, an intra-individual analysis is performed on sequence information of target nucleic acids and sequence information of reference nucleic acids. As described herein, the sequence information of target nucleic acids and sequence information of reference nucleic acids are generated by performing one or more assays (e.g., assay 120A and/or assay 120B).

The intra-individual analysis involves combining the sequence information of target nucleic acids and sequence information of reference nucleic acids to generate a signal informative for determining presence or absence of a health condition. Here, the step of combining the sequence information of target nucleic acids and sequence information of reference nucleic acids can be performed by the signal generation module 210 shown in FIG. 2A.

In various embodiments, combining the sequence information of target nucleic acids and sequence information of reference nucleic acids involves differentiating between signatures present or absent in the sequence information of target nucleic acids and signatures present or absent in the sequence information of the reference nucleic acids. For example, if particular signatures are present in the sequence information of target nucleic acids, and the signatures are also present in the sequence information of reference nucleic acids, the signatures in both the target nucleic acids and reference nucleic acids may represent baseline biological signatures. Thus, these signatures may be excluded from the resulting signal informative of determining presence or absence of the health condition. As another example, if particular signatures are present in the sequence information of target nucleic acids, but those signatures are absent in the sequence information of reference nucleic acids, the signatures may not be baseline biological signatures. Thus, these signatures may be included in the resulting signal informative of determining presence or absence of the health condition.

In various embodiments, combining the sequence information of the target nucleic acids and the sequence information of the reference nucleic acids includes aligning the sequence information of the target nucleic acids and the sequence information of the reference nucleic acids. For example, aligning the sequence information involves aligning sequences of a plurality of pre-selected genomic sites for the target nucleic acids and sequences of the same or overlapping plurality of pre-selected genomic sites for the reference nucleic acids.

In various embodiments, both the sequence information of the target nucleic acids and the sequence information of the reference nucleic acids are aligned to a reference genome library (e.g., a reference assembly) with known sequences. Therefore, sequence information of the target nucleic acids are aligned to the sequence information of the reference nucleic acids via the reference genome library. In various embodiments, the sequence information of the target nucleic acids is aligned directly with the sequence information of the reference nucleic acids. In such embodiments, a reference genome library need not be used.

In various embodiments, combining the sequence information of the target nucleic acids and the sequence information of the reference nucleic acids includes determining a difference between the sequence information of the target nucleic acids to the sequence information of the reference nucleic acids.

As disclosed herein, target nucleic acids can include cell-free DNA in the blood that originates from a cancer cell. Reference nucleic acids may be, for example, cellular genomic DNA or germline DNA derived from a non-cancerous cells, e.g., peripheral blood mononuclear cells (PBMCs) or polymorphonuclear cells. PBMCs refer to any peripheral blood cell having a round nucleus, examples of which include, but are not limited to: lymphocytes (T cells, B cells, natural killer cells, and monocytes). Polymorphonuclear cells refer to cells with multiple nuclei (e.g., two or three), examples of which include granulocytes, eosinophils, basophils, neutrophils, and mast cells. Thus, in various embodiments, combining the sequence information from the target nucleic acids and the sequence information from the reference nucleic acids includes determining a difference between the sequence information from the cell-free DNA in the blood that originates from a cancer cell and the sequence information from the germline DNA derived from a non-cancerous cells, e.g., peripheral blood mononuclear cells (PBMCs) or polymorphonuclear cells.

In various embodiments, sequence information from the target nucleic acids can include phased sequencing information (e.g., haplotype sequencing information from either the maternal or paternal chromosome) derived from cell-free DNA in the blood that originates from a cancer cell. In various embodiments, sequence information from the reference nucleic acids can include phased sequencing information (e.g., haplotype sequencing information from either the maternal or paternal chromosome) derived from germline DNA (e.g., from PBMCs or polymorphonuclear cells). Thus, in various embodiments, combining the sequence information from the target nucleic acids and the sequence information from the reference nucleic acids includes determining a difference between the phased sequencing information derived from cell-free DNA in the blood that originates from a cancer cell and the phased sequencing information derived from germline DNA (e.g., from PBMCs or polymorphonuclear cells). For example, combining the sequence information from the target nucleic acids and the sequence information from the reference nucleic acids includes determining a difference between the phased sequencing information corresponding to a maternal chromosome derived from cell-free DNA in the blood that originates from a cancer cell and the phased sequencing information corresponding to a maternal chromosome derived from germline DNA (e.g., from PBMCs or polymorphonuclear cells). As another example, combining the sequence information from the target nucleic acids and the sequence information from the reference nucleic acids includes determining a difference between the phased sequencing information corresponding to a paternal chromosome derived from cell-free DNA in the blood that originates from a cancer cell and the phased sequencing information corresponding to a paternal chromosome derived from germline DNA (e.g., from PBMCs or polymorphonuclear cells).

In various embodiments, differences between the sequence information of the target nucleic acids and the sequence information of the reference nucleic acids are performed on a per-position basis. For example, at a first position of a genomic site, the difference between the sequence information of the target nucleic acids at the first position and the sequence information of the reference nucleic acid at the same first position is determined. The process can then be further repeated for additional positions (e.g., for additional positions across the plurality of genomic sites). In various embodiments, the differences are determined on a per-position basis if the sequence information of the target nucleic acids and reference nucleic acids were generated using a sequencing assay (e.g., next generation sequencing) which provides base-level resolution of the sequences.

In various embodiments, differences between the sequence information of the target nucleic acids and the sequence information of the reference nucleic acids are performed on a per-CGI basis. For example, at a first CGI of a genomic site, the difference between the sequence information of the target nucleic acids at the first CGI and the sequence information of the reference nucleic acid at the same CGI or overlapping portion of the first CGI is determined. The process can then be further repeated for additional CGIs (e.g., for additional CGIs across the plurality of genomic sites). In various embodiments, the differences are determined on a per-CGI basis if the sequence information of the target nucleic acids and reference nucleic acids were generated using a quantitative assay (e.g., qPCR assay).

In various embodiments, differences between the sequence information of the target nucleic acids and the sequence information of the reference nucleic acids are performed on a per-allele basis. For example, at a first allele of a genomic site, the difference between the sequence information of the target nucleic acids at the first allele and the sequence information of the reference nucleic acid at the same allele or overlapping portion of the first allele is determined. The process can then be further repeated for additional alleles (e.g., for additional alleles across the plurality of genomic sites). In various embodiments, the differences are determined on a per-allele basis if the sequence information of the target nucleic acids and reference nucleic acids were generated using a quantitative assay (e.g., qPCR assay or allele-specific PCR assay).

Reference is now made to FIG. 2B, which depicts an example combining of sequence information of target nucleic acids and reference nucleic acids to generate a signal informative for a health condition, in accordance with an embodiment. The sequence information of the target nucleic acids and the sequence information of the reference nucleic acids include methylation statuses across a plurality of genomic sites. FIG. 2B shows an example genomic site in which nucleotide bases may be differentially methylated in the target nucleic acid and the reference nucleic acid. In various embodiments, combining sequence information of target nucleic acids and reference nucleic acids involves combining methylation statuses of one or more CpG sites of the target nucleic acids and reference nucleic acids. For example, combining methylation statuses of one or more CpG sites can involve subtracting a methylation status of the reference nucleic acid from the methylation status of the target nucleic acid.

The term “subtracting” is used in the context of methylation statuses of a target nucleic acid and reference nucleic acid. For example, at a particular CpG site in each of the target nucleic acid and reference nucleic acid, if the methylation status of the target nucleic acid and reference nucleic acid are the same (e.g., both methylated or both non-methylated), then subtracting the methylation status of the reference nucleic acid from the methylation status of the target nucleic acid results in a non-methylated CpG site in the resulting cancer signal. This scenario arises when a methylated CpG site arises from a germline source and therefore, may not be informative of cancer. In contrast, at a particular CpG site in each of the target nucleic acid and reference nucleic acid, the target nucleic acid and reference nucleic acid may be differentially methylated. For example, for a particular CpG site, assume the target nucleic acid includes a methylated CpG site and the reference nucleic acid includes a non-methylated CpG site. In this scenario, subtracting the methylation status of the reference nucleic acid from the methylation status of the target nucleic acid results in a methylated CpG site in the resulting cancer signal. This scenario arises when a methylated CpG site arises from a cancer source (and is not present in the germline). Thus, the methylated CpG site may be informative of cancer. As another example, for a particular CpG site, assume the target nucleic acid includes a non-methylated CpG site and the reference nucleic acid includes a methylated CpG site. In this scenario, subtracting the methylation status of the reference nucleic acid from the methylation status of the target nucleic acid results in a non-methylated CpG site in the resulting cancer signal.

As a specific example, as shown in FIG. 2B, the nucleotide base at the second position is methylated (as represented by the presence of a cytosine base which arises following bisulfite conversion) in both the target nucleic acid and the reference nucleic acid. Given that the methylation at the second position occurs in both the target nucleic acid and the reference nucleic acid, this may be a baseline biological signature. Thus, by subtracting the methylation status at the second position of the reference nucleic acid from the methylation status at the second position of the target nucleic acid, the resulting cancer signal includes a non-methylated cytosine at the second position.

Conversely, the target nucleic acid may additionally be methylated at the sixth position and the ninth position, whereas the reference nucleic acid is unmethylated at the sixth position and the ninth position. Here, given that the reference nucleic acid is not methylated at the sixth and ninth position, the presence of the methylated nucleotide bases in the target nucleic acid may represent signatures that are informative of presence or absence of the health condition. Thus, by subtracting the methylation status at the sixth position of the reference nucleic acid from the methylation status at the sixth position of the target nucleic acid, the resulting cancer signal includes a methylated cytosine at the sixth position. Similarly, by subtracting the methylation status at the ninth position of the reference nucleic acid from the methylation status at the ninth position of the target nucleic acid, the resulting cancer signal includes a methylated cytosine at the ninth position.

Additionally, at the eleventh nucleotide position, the target nucleic acid is unmethylated whereas the reference nucleic acid is methylated. Here, the methylation of the reference nucleic acid can be interpreted as a baseline biological signature. In this example, by subtracting the methylation status at the eleventh position of the reference nucleic acid from the methylation status at the eleventh position of the target nucleic acid, the resulting cancer signal includes a non-methylated cytosine at the eleventh position.

The differences between the methylation status at each position of the target nucleic acid and the reference nucleic acid can represent the cancer signal. As shown in FIG. 2B, the cancer signal includes methylation statuses at the genomic site, wherein the sixth and ninth position are methylated. Thus, the cancer signal includes signatures from the target nucleic acids that are likely informative of the health condition (e.g., methylated statuses of the sixth and ninth nucleotide bases), and further excludes baseline biological signatures (e.g., baseline biological signatures present in reference nucleic acids such as methylated statuses of the second and eleventh nucleotide bases).

In various embodiments, referring to FIG. 2B, the target nucleic acid and the reference nucleic acid represent signatures from a common source, such as a paternal chromosome or a maternal chromosome. For example, the target nucleic acid and the reference nucleic acid may represent signatures corresponding to a paternal chromosome. As another example, the target nucleic acid and the reference nucleic acid may represent signatures corresponding to a paternal chromosome. Ensuring that the target nucleic acid and reference nucleic acid are from a common source can avoid inadvertently capturing germline differences that may be present in different sources in a cancer signal. For example, the maternal chromosome and paternal chromosome may include differing germline sequences. If the target nucleic acid and reference nucleic acid are signatures from different sources, then the germline differences may be inadvertently captured in the resulting cancer signal. In various embodiments, the target nucleic acid and the reference nucleic acid need not have been previously identified as specifically corresponding to a paternal chromosome or maternal chromosome; rather, it may be sufficient to have identified that the target nucleic acid and the reference acid correspond to a common or different source. If a target nucleic acid and a reference nucleic acid are from a common source, then the sequence information from the target nucleic acids and the sequence information from the reference nucleic acids are combined (as shown in FIG. 2B). If a target nucleic acid and a reference nucleic acid are from different sources, then the sequence information from the target nucleic acids and the sequence information from the reference nucleic acids are not combined.

In various embodiments, referring to FIG. 2B, the target nucleic acid and the reference nucleic acid can be signatures generated via two different types of sequencing. For example, different types of sequencing can include shallow sequencing and deep sequencing. In particular embodiments, the target nucleic acid is a signature generated via deep sequencing and the reference nucleic acid is a signature generated via shallow sequencing. Given that the goal of the cancer signal is to retain signatures of rare cancer events, the reference nucleic acid representing a baseline signature generated via shallow sequencing may not include signatures of these rare cancer events. In contrast, the target nucleic acid generated via deep sequencing can include signatures of these rare cancer events. Therefore, by combining the target nucleic acid generated via deep sequencing and the reference nucleic acid generated via shallow sequencing, the resulting cancer signal retains the signatures of rare cancer events.

In some embodiments, the target nucleic acid and the reference nucleic acid may both originate from cell-free DNA (e.g., cell-free DNA from a liquid biopsy, which may include a mixture of nucleic acids from non-cancerous cells and nucleic acids from cancerous cells). Here, since cell-free tumor DNA is rare within the cell-free DNA mixture, shallow sequencing may not capture signatures from the cell-free tumor DNA due to the low probability of the sequencing reaction occurring on a cell-free tumor DNA fragment. In contrast, through deep sequencing, additional reads of a given target region increases the probability that a cell-free tumor DNA fragment will be encountered and sequenced. Therefore, the signature of the reference nucleic acid can be generated via shallow sequencing from cancer cells, but only contains baseline signatures and not signatures of rare cancer events. In contrast, the signature of the target nucleic acid can be generated via deep sequencing from cancer cells, and contains both baseline signatures and signatures of rare cancer events.

In various embodiments, referring to FIG. 2B, the target nucleic acid and the reference nucleic acid represent 1) signatures from a common source, such as a paternal chromosome or a maternal chromosome and 2) signatures generated via two different types of sequencing. For example, the target nucleic acid and the reference nucleic acid represent signatures from a paternal or maternal chromosome and furthermore, the target nucleic acid is a signature generated via deep sequencing and the reference nucleic acid is a signature generated via shallow sequencing. Thus, the resulting cancer signal can represent a more informative cancer signature.

The intra-individual analysis may further involve analyzing the signal representing the combination of the sequence information of the target nucleic acids and the sequence information of the reference nucleic acids to determine whether a health condition is present or absent in the individual. Here, the step of analyzing the signal to determine presence of absence of the health condition can be performed by the signal analysis module 220 shown in FIG. 2A. In various embodiments, a machine learning model is deployed to analyze a signal informative for determining presence or absence of the health condition. The machine learning model analyzes the signal, which represents the difference between epigenetic statuses (e.g., methylation statuses) of the plurality of genomic sites of target nucleic acids and epigenetic statuses (e.g., methylation statuses) of the plurality of genomic sites of reference nucleic acids. Therefore, trained machine learning models analyze the signal across the plurality of genomic sites to output a prediction as to whether the individual has a presence or absence of the health condition. In particular embodiments, the machine learning model analyzes the signal, which represents the difference between epigenetic statuses (e.g., methylation statuses) of phased sequencing information (e.g., methylation statuses of genomic sites derived from common sources, such as a maternal or paternal chromosome) of target nucleic acids and phase sequencing information of reference nucleic acids. Therefore, trained machine learning models analyze the signal across the genomic sites in the phased sequencing information to output a prediction as to whether the individual has a presence or absence of the health condition.

In particular embodiments, machine learning models analyze methylation statuses of a plurality of genomic sites in cell-free DNA to generate predictions. The methylation statuses can correspond to a set of cancer informative CpG islands (CGIs), wherein the cancer informative CGIs are selected from a group consisting of a ranked set of candidate CGIs. In various embodiments, a machine learning model analyzes methylation statuses for at least 50 CGIs. In various embodiments, a machine learning model analyzes methylation statuses for at least 100 CGIs. In various embodiments, a machine learning model analyzes methylation statuses for at least 150 CGIs. In various embodiments, a machine learning model analyzes methylation statuses for at least 200 CGIs. In various embodiments, a machine learning model analyzes methylation statuses for at least 250 CGIs. In various embodiments, a machine learning model analyzes methylation statuses for at least 300 CGIs. In various embodiments, a machine learning model analyzes methylation statuses for at least 400 CGIs. In various embodiments, a machine learning model analyzes methylation statuses for at least 500 CGIs. In various embodiments, a machine learning model analyzes methylation statuses for at least 600 CGIs. In various embodiments, a machine learning model analyzes methylation statuses for at least 700 CGIs. In various embodiments, a machine learning model analyzes methylation statuses for at least 800 CGIs. In various embodiments, a machine learning model analyzes methylation statuses for at least 900 CGIs. In various embodiments, a machine learning model analyzes methylation statuses for at least 1000 CGIs. In various embodiments, a machine learning model analyzes methylation statuses for at least 2500 CGIs. In various embodiments, a machine learning model analyzes methylation statuses for at least 5000 CGIs. In various embodiments, a machine learning model analyzes methylation statuses for at least 7500 CGIs. In various embodiments, a machine learning model analyzes methylation statuses for at least 10000 CGIs. In various embodiments, a machine learning model analyzes methylation statuses for at least 15000 CGIs. In various embodiments, a machine learning model analyzes methylation statuses for at least 20000 CGIs. In various embodiments, a machine learning model analyzes methylation statuses for at least 25000 CGIs.

In various embodiments, a machine learning model analyzes methylation statuses for CGIs across the whole genome. For example, a machine learning model may be implemented to analyze sequencing data generated from whole genome sequencing (e.g., whole genome bisulfite sequencing).

In particular embodiments, the intra-individual analysis further reveals, for an individual predicted to have a presence of the health condition, a tissue of origin of the health condition. The intra-individual analysis may identify a tissue of origin of the health condition according to the methylation statuses of the cancer informative CGIs. For example, particular methylation patterns across the cancer informative CGIs are attributable to certain tissues, examples of which include the nervous tissue (e.g., brain, spinal cord, nerves), muscle tissue (cardiac muscle, smooth muscle, skeletal muscle), epithelial tissue (e.g., GI tract lining, skin), and connective tissue (e.g., fat, bone, tendon, and ligaments). As a particular example, in patients with brain cancer, a first set of CGIs may be frequently methylated. Therefore, if a similar methylation pattern is observed across the first set of CGIs for an individual, the intra-individual analysis can identify that the individual has cancer, and furthermore, that the cancer is localized to the brain.

### Example Methods for Conducting an Intra-Individual Analysis

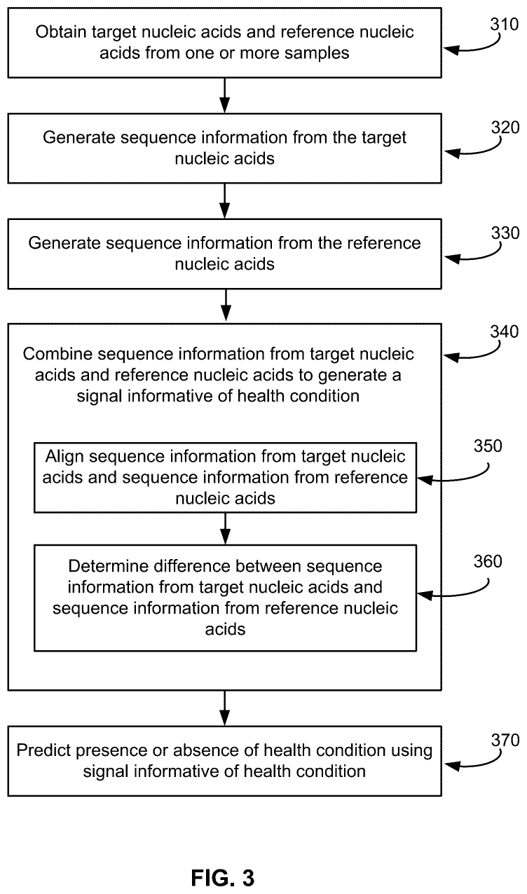

FIG. 3 shows an example flow process involving an intra-individual analysis, in accordance with an embodiment. Step 310 involves obtaining target nucleic acids and reference nucleic acids from one or more samples.

Step 320 involves generating sequence information from the target nucleic acids. Here, sequence information from the target nucleic acids may include signatures informative for determining presence or absence of the health condition, but it may also include baseline biological signatures that are present irrespective of whether the nucleic acids originate from a diseased source or a non-diseased source. Step 330 involves generating sequence information from the reference nucleic acids. Sequence information of the reference nucleic acids include baseline biological signatures, which are less informative for determining presence or absence of the health condition in comparison to sequence information of the target nucleic acids.

Step 340 involves combining sequence information from target nucleic acids and sequence information from reference nucleic acids to generate a signal informative for determining presence or absence of the health condition. As shown in FIG. 3, step 340 can include both steps 350 and 360. Step 350 involves aligning sequence information from target nucleic acids with sequence information from reference nucleic acids. Step 360 involves determining a difference between sequence information from target nucleic acids and sequence information from reference nucleic acids. In various embodiments, step 360 involves determining a difference on a per-position basis.

Step 370 involves predicting presence or absence of a health condition using the signal informative of the health condition. Thus, if the individual is determined to have presence of the health condition, the individual can be provided treatment to prophylactically or therapeutically treat the health condition.

### Additional Example Methods for Conducting an Intra-Individual Analysis

Disclosed herein are additional example methods for conducting an intra-individual analysis. Referring again to FIG. 3, additional example methods may include additional steps under step 340 which involves combining sequence information from target nucleic acids and reference nucleic acids to generate a signal informative of health condition.

For example, referring to FIG. 3, step 310 involves obtaining target nucleic acids and reference nucleic acids from one or more samples. Step 320 involves generating sequence information from the target nucleic acids. Here, sequence information from the target nucleic acids may include signatures informative for determining presence or absence of the health condition, but it may also include baseline biological signatures that are present irrespective of whether the nucleic acids originate from a diseased source or a non-diseased source. Step 330 involves generating sequence information from the reference nucleic acids. Sequence information of the reference nucleic acids include baseline biological signatures, which are less informative for determining presence or absence of the health condition in comparison to sequence information of the target nucleic acids.

Step 340 involves combining sequence information from target nucleic acids and sequence information from reference nucleic acids to generate a signal informative for determining presence or absence of the health condition. In various embodiments, step 340 may include a first substep of determining ratios of methylation levels amongst two or more CpG sites (e.g., methylation levels amongst two or more CpG sites located in CpG islands or portions of CpG islands shown in Tables 1-4) in the target nucleic acids. In some embodiments, the two or more CpG sites are in a common CpG island. In some embodiments, the two or more CpG sites are in different CpG islands. In some embodiments, a subset of the two or more CpG sites are in a common CpG island, and a second subset of the two or more CpG sites are in at least a different CpG island.

In some scenarios, the first substep may involve determining a ratio of methylation levels of two CpG sites. For example, given a first CpG site and a second CpG site, the ratio of methylation levels between the first and second CpG site can be a number of reads containing a methylated first CpG site divided by a number of reads containing a methylated second CpG site. As another example, given a first CpG site and a second CpG site, the ratio of methylation levels between the first and second CpG site can be a proportion of reads containing the first CpG site that are methylated divided by a proportion of reads containing the second CpG site that are methylated. In some scenarios, the first substep may involve determining ratios of methylation levels of three CpG sites, of four CpG sites, of five CpG sites, of six CpG sites, of seven CpG sites, of eight CpG sites, of nine CpG sites, of ten CpG sites, of eleven CpG sites, of twelve CpG sites, of thirteen CpG sites, or fourteen CpG sites, of fifteen CpG sites, of twenty CpG sites, of thirty CpG sites, of forty CpG sites, of fifty CpG sites, of sixty CpG sites, of seventy CpG sites, of eighty CpG sites, of ninety CpG sites, or a hundred CpG sites.

Referring back to step 340, a second substep can be step 350, which involves aligning the sequence information from target nucleic acids and sequence information from reference nucleic acids. The third substep can be step 360 which involves determining a difference between the sequence information from target nucleic acids and sequence information from reference nucleic acids. As described herein, determining the difference can include subtracting a methylation status of the reference nucleic acid from the methylation status of the target nucleic acid, thereby generating a signal that includes limited or no baseline signatures.

A fourth substep may involve determining additional ratios of methylation levels amongst two or more CpG sites (e.g., methylation levels amongst two or more CpG sites located in CpG islands or portions of CpG islands shown in Tables 1-4) in the signal that includes limited or no baseline signatures. Here, the fourth substep may involve determining additional ratios of methylation levels amongst the same CpG sites that were analyzed in the first substep. For example, if the first substep involved determining a ratio of methylation levels between a first CpG site and a second CpG site in the target nucleic acid, the fourth substep further involves determining an additional ratio of methylation levels between the same first CpG site and the same second CpG site in the signal that includes limited or no baseline signature (generated at step 360). In some scenarios, the fourth substep may involve determining additional ratios of methylation levels of three CpG sites, of four CpG sites, of five CpG sites, of six CpG sites, of seven CpG sites, of eight CpG sites, of nine CpG sites, of ten CpG sites, of eleven CpG sites, of twelve CpG sites, of thirteen CpG sites, or fourteen CpG sites, of fifteen CpG sites, of twenty CpG sites, of thirty CpG sites, of forty CpG sites, of fifty CpG sites, of sixty CpG sites, of seventy CpG sites, of eighty CpG sites, of ninety CpG sites, or a hundred CpG sites.

A fifth substep involves comparing the ratios of methylation levels amongst two or more CpG sites generated from target nucleic acids at the first substep with the additional ratios of methylation levels amongst the same two or more CpG sites generated from the signal that includes limited or no baseline signatures. For example, assume the first substep involved determining a ratio of methylation levels between a first CpG site and a second CpG site in the target nucleic acid, and the four substep involved determining an additional ratio of methylation levels between the same first CpG site and the same second CpG site in the signal that includes limited or no baseline signature (generated at step 360). Thus, this fifth substep involves comparing the two ratios. In various embodiments, the change in the two ratios (e.g., from the ratio to the additional ratio) represents a signal informative of the health condition. For example, in some embodiments, the ratio of methylation levels between a first CpG site and a second CpG site may increase as a result of the removal of the baseline signatures (as conducted in step 360). Thus, the increase in the ratio can be a signal informative of presence or absence of the health condition. As another example, in some embodiments, the ratio of methylation levels between a first CpG site and a second CpG site may decrease as a result of the removal of the baseline signatures (as conducted in step 360). Thus, the decrease in the ratio can be a signal informative of presence or absence of the health condition. In various embodiments, if the removal of baseline signatures results in limited or no change in the ratio, then the resulting signal can be informative of an absence of the health condition. In various embodiments, if the removal of baseline signatures results in significant change in the ratio, then the resulting signal can be informative of a presence of the health condition. In various embodiments, a “significant change” can refer to at least a 1.5-fold, at least a 1.75 fold, at least a 2.0 fold, at least a 2.5 fold, at least a 3 fold, at least a 4 fold, at least a 5 fold, at least a 6 fold, at least a 7 fold, at least a 8 fold, at least a 9 fold, or at least 10 fold increase or decrease in the ratio as a result of the removal of the baseline signatures.

Although this description specifically references a single ratio for two CpG sites, the description can be similarly applied to more ratios. For example, there may be R different ratios determined for different CpG sites from the target nucleic acids (at the first substep) and similarly, R different ratios determined for the same CpG sites from the signal that includes limited or no baseline signatures (at the fourth substep). Thus, comparing the R different ratios before and after the removal of the baseline signatures determines the changes in the R different ratios. The combination of the changes in the R different ratios can represent the signal informative of presence or absence of the health condition.

Returning to step 370, it involves predicting presence or absence of a health condition using the signal informative of the health condition. Thus, if the individual is determined to have presence of the health condition, the individual can be provided treatment to prophylactically or therapeutically treat the health condition.

Health Conditions

The disclosure provides methods for performing an intra-individual analysis to determine a presence or absence of a health condition in a patient. In various embodiments, the patient may be suspected of having a health condition, but may not have been previously identified as having a health condition. In various embodiments, the patient is healthy and is not yet suspected of having a health condition.

In various embodiments, the health condition can be a disease or disorder. Examples of diseases and/or disorders can include, for example, a cancer, inflammatory disease, neurodegenerative disease, autoimmune disorder, neuromuscular disease, metabolic disorder (e.g., diabetes), cardiac disease, or fibrotic disease (e.g., idiopathic pulmonary fibrosis).

In particular embodiments, the health condition is a cancer. In various embodiments, the cancer is an early stage cancer. In various embodiments, the cancer is a preclinical phase cancer. In various embodiments, the cancer is a stage I cancer. In various embodiments, the cancer is a stage II cancer.

In various embodiments, the cancer is any of an acute lymphoblastic leukemia, acute myeloid leukemia, adrenocortical carcinoma, soft tissue sarcoma, lymphoma, anal cancer, gastrointestinal cancer, brain cancer, skin cancer, bile duct cancer, bladder cancer, bone cancer, breast cancer, lung cancer, cardiac cancer, central nervous system cancer, cervical cancer, chronic lymphocytic leukemia, chronic myelogenous leukemia, chronic myeloproliferative neoplasms, colorectal cancer, uterine cancer, esophageal cancer, head and neck cancer, eye cancer, fallopian tube cancer, gallbladder cancer, gastric cancer, germ cell tumor, gestational trophoblastic cancer, hairy cell leukemia, liver cancer, Hodgkin lymphoma, intraocular melanoma, pancreatic cancer, kidney cancer, leukemia, mesothelioma, metastatic cancer, mouth cancer, multiple endocrine neoplasia syndromes, multiple myeloma neoplasms, myelodysplastic neoplasms, ovarian cancer, parathyroid cancer, penile cancer, pheochromocytoma, pituitary cancer, plasma cell neoplasm, primary peritoneal cancer, prostate cancer, rectal cancer, retinoblastoma, sarcoma, small intestine cancer, testicular cancer, throat cancer, thymoma and thymic carcinoma, thyroid cancer, urethral cancer, uterine cancer, vaginal cancer, and vulvar cancer.

In various embodiments, the inflammatory disease can be any one of acute respiratory distress syndrome (ARDS), acute lung injury (ALI), alcoholic liver disease, allergic inflammation of the skin, lungs, and gastrointestinal tract, allergic rhinitis, ankylosing spondylitis, asthma (allergic and non-allergic), atopic dermatitis (also known as atopic eczema), atherosclerosis, celiac disease, chronic obstructive pulmonary disease (COPD), chronic respiratory distress syndrome (CRDS), colitis, dermatitis, diabetes, eczema, endocarditis, fatty liver disease, fibrosis (e.g., idiopathic pulmonary fibrosis, scleroderma, kidney fibrosis, and scarring), food allergies (e.g., allergies to peanuts, eggs, dairy, shellfish, tree nuts, etc.), gastritis, gout, hepatic steatosis, hepatitis, inflammation of body organs including joint inflammation including joints in the knees, limbs or hands, inflammatory bowel disease (IBD) (including Crohn's disease or ulcerative colitis), intestinal hyperplasia, irritable bowel syndrome, juvenile rheumatoid arthritis, liver disease, metabolic syndrome, multiple sclerosis, myasthenia gravis, neurogenic lung edema, nephritis (e.g., glomerular nephritis), non-alcoholic fatty liver disease (NAFLD) (including non-alcoholic steatosis and non-alcoholic steatohepatitis (NASH)), obesity, prostatitis, psoriasis, psoriatic arthritis, rheumatoid arthritis (RA), sarcoidosis sinusitis, splenitis, seasonal allergies, sepsis, systemic lupus erythematosus, uveitis, and UV-induced skin inflammation.

In various embodiments, the neurodegenerative disease can be any one of Alzheimer's disease, Parkinson's disease, traumatic CNS injury, Down Syndrome (DS), glaucoma, amyotrophic lateral sclerosis (ALS), frontotemporal dementia (FTD), and Huntington's disease. In addition, the neurodegenerative disease can also include Absence of the Septum Pellucidum, Acid Lipase Disease, Acid Maltase Deficiency, Acquired Epileptiform Aphasia, Acute Disseminated Encephalomyelitis, ADHD, Adie's Pupil, Adie's Syndrome, Adrenoleukodystrophy, Agenesis of the Corpus Callosum, Agnosia, Aicardi Syndrome, AIDS, Alexander Disease, Alper's Disease, Alternating Hemiplegia, Anencephaly, Aneurysm, Angelman Syndrome, Angiomatosis, Anoxia, Antiphosphipid Syndrome, Aphasia, Apraxia, Arachnoid Cysts, Arachnoiditis, Arnold-Chiari Malformation, Arteriovenous Malformation, Asperger Syndrome, Ataxia, Ataxia Telangiectasia, Ataxias and Cerebellar or Spinocerebellar Degeneration, Autism, Autonomic Dysfunction, Barth Syndrome, Batten Disease, Becker's Myotonia, Behcet's Disease, Bell's Palsy, Benign Essential Blepharospasm, Benign Focal Amyotrophy, Benign Intracranial Hypertension, Bernhardt-Roth Syndrome, Binswanger's Disease, Blepharospasm, Bloch-Sulzberger Syndrome, Brachial Plexus Injuries, Bradbury-Eggleston Syndrome, Brain or Spinal Tumors, Brain Aneurysm, Brain injury, Brown-Sequard Syndrome, Bulbospinal Muscular Atrophy, Cadasil, Canavan Disease, Causalgia, Cavernomas, Cavernous Angioma, Central Cord Syndrome, Central Pain Syndrome, Central Pontine Myelinolysis, Cephalic Disorders, Ceramidase Deficiency, Cerebellar Degeneration, Cerebellar Hypoplasia, Cerebral Aneurysm, Cerebral Arteriosclerosis, Cerebral Atrophy, Cerebral Beriberi, Cerebral Gigantism, Cerebral Hypoxia, Cerebral Palsy, Cerebro-Oculo-Facio-Skeletal Syndrome, Charcot-Marie-Tooth Disease, Chiari Malformation, Chorea, Chronic Inflammatory Demyelinating Polyneuropathy (CIDP), Coffin Lowry Syndrome, Colpocephaly, Congenital Facial Diplegia, Congenital Myasthenia, Congenital Myopathy, Corticobasal Degeneration, Cranial Arteritis, Craniosynostosis, Creutzfeldt-Jakob Disease, Cumulative Trauma Disorders, Cushing's Syndrome, Cytomegalic Inclusion Body Disease, Dancing Eyes-Dancing Feet Syndrome, Dandy-Walker Syndrome, Dawson Disease, Dementia, Dementia With Lewy Bodies, Dentate Cerebellar Ataxia, Dentatorubral Atrophy, Dermatomyositis, Developmental Dyspraxia, Devic's Syndrome, Diabetic Neuropathy, Diffuse Sclerosis, Dravet Syndrome, Dysautonomia, Dysgraphia, Dyslexia, Dysphagia, Dyssynergia Cerebellaris Myoclonica, Dystonias, Early Infantile Epileptic Encephalopathy, Empty Sella Syndrome, Encephalitis, Encephalitis Lethargica, Encephaloceles, Encephalopathy, Encephalotrigeminal Angiomatosis, Epilepsy, Erb-Duchenne and Dejerine-Klumpke Palsies, Erb's Palsy, Essential Tremor, Extrapontine Myelinolysis, Fabry Disease, Fahr's Syndrome, Fainting, Familial Dysautonomia, Familial Hemangioma, Familial Periodic Paralyzes, Familial Spastic Paralysis, Farber's Disease, Febrile Seizures, Fibromuscular Dysplasia, Fisher Syndrome, Floppy Infant Syndrome, Foot Drop, Friedreich's Ataxia, Frontotemporal Dementia, Gangliosidoses, Gaucher's Disease, Gerstmann's Syndrome, Gerstmann-Straussler-Scheinker Disease, Giant Cell Arteritis, Giant Cell Inclusion Disease, Globoid Cell Leukodystrophy, Glossopharyngeal Neuralgia, Glycogen Storage Disease, Guillain-Barre Syndrome, Hallervorden-Spatz Disease, Head Injury, Hemicrania Continua, Hemifacial Spasm, Hemiplegia Alterans, Hereditary Neuropathy, Hereditary Spastic Paraplegia, Heredopathia Atactica Polyneuritiformis, Herpes Zoster, Herpes Zoster Oticus, Hirayama Syndrome, Holmes-Adie syndrome, Holoprosencephaly, HTLV-1 Associated Myelopathy, Hughes Syndrome, Huntington's Disease, Hydranencephaly, Hydrocephalus, Hydromyelia, Hypernychthemeral Syndrome, Hypersomnia, Hypertonia, Hypotonia, Hypoxia, Immune-Mediated Encephalomyelitis, Inclusion Body Myositis, Incontinentia Pigmenti, Infantile Hypotonia, Infantile Neuroaxonal Dystrophy, Infantile Phytanic Acid Storage Disease, Infantile Refsum Disease, Infantile Spasms, Inflammatory Myopathies, Iniencephaly, Intestinal Lipodystrophy, Intracranial Cysts, Intracranial Hypertension, Isaac's Syndrome, Joubert syndrome, Kearns-Sayre Syndrome, Kennedy's Disease, Kinsbourne syndrome, Kleine-Levin Syndrome, Klippel-Feil Syndrome, Klippel-Trenaunay Syndrome (KTS), Kluver-Bucy Syndrome, Korsakoffs Amnesic Syndrome, Krabbe Disease, Kugelberg-Welander Disease, Kuru, Lambert-Eaton Myasthenic Syndrome, Landau-Kleffner Syndrome, Lateral Medullary Syndrome, Learning Disabilities, Leigh's Disease, Lennox-Gastaut Syndrome, Lesch-Nyhan Syndrome, Leukodystrophy, Levine-Critchley Syndrome, Lewy Body Dementia, Lipid Storage Diseases, Lipoid Proteinosis, Lissencephaly, Locked-In Syndrome, Lou Gehrig's Disease, Lupus, Lyme Disease, Machado-Joseph Disease, Macrencephaly, Melkersson-Rosenthal Syndrome, Meningitis, Menkes Disease, Meralgia Paresthetica, Metachromatic Leukodystrophy, Microcephaly, Migraine, Miller Fisher Syndrome, Mini-Strokes, Mitochondrial Myopathies, Motor Neuron Diseases, Moyamoya Disease, Mucolipidoses, Mucopolysaccharidoses, Multiple sclerosis (MS), Multiple System Atrophy, Muscular Dystrophy, Myasthenia Gravis, Myoclonus, Myopathy, Myotonia, Narcolepsy, Neuroacanthocytosis, Neurodegeneration with Brain Iron Accumulation, Neurofibromatosis, Neuroleptic Malignant Syndrome, Neurosarcoidosis, Neurotoxicity, Nevus Cavernosus, Niemann-Pick Disease, Non 24 Sleep Wake Disorder, Normal Pressure Hydrocephalus, Occipital Neuralgia, Occult Spinal Dysraphism Sequence, Ohtahara Syndrome, Olivopontocerebellar Atrophy, Opsoclonus Myoclonus, Orthostatic Hypotension, O'Sullivan-McLeod Syndrome, Overuse Syndrome, Pantothenate Kinase-Associated Neurodegeneration, Paraneoplastic Syndromes, Paresthesia, Parkinson's Disease, Paroxysmal Choreoathetosis, Paroxysmal Hemicrania, Parry-Romberg, Pelizaeus-Merzbacher Disease, Perineural Cysts, Periodic Paralyzes, Peripheral Neuropathy, Periventricular Leukomalacia, Pervasive Developmental Disorders, Pinched Nerve, Piriformis Syndrome, Plexopathy, Polymyositis, Pompe Disease, Porencephaly, Postherpetic Neuralgia, Postinfectious Encephalomyelitis, Post-Polio Syndrome, Postural Hypotension, Postural Orthostatic Tachyardia Syndrome (POTS), Primary Lateral Sclerosis, Prion Diseases, Progressive Multifocal Leukoencephalopathy, Progressive Sclerosing Poliodystrophy, Progressive Supranuclear Palsy, Prosopagnosia, Pseudotumor Cerebri, Ramsay Hunt Syndrome I, Ramsay Hunt Syndrome II, Rasmussen's Encephalitis, Reflex Sympathetic Dystrophy Syndrome, Refsum Disease, Refsum Disease, Repetitive Motion Disorders, Repetitive Stress Injuries, Restless Legs Syndrome, Retrovirus-Associated Myelopathy, Rett Syndrome, Reye's Syndrome, Rheumatic Encephalitis, Riley-Day Syndrome, Saint Vitus Dance, Sandhoff Disease, Schizencephaly, Septo-Optic Dysplasia, Shingles, Shy-Drager Syndrome, Sjogren's Syndrome, Sleep Apnea, Sleeping Sickness, Sotos Syndrome, Spasticity, Spinal Cord Infarction, Spinal Cord Injury, Spinal Cord Tumors, Spinocerebellar Atrophy, Spinocerebellar Degeneration, Stiff-Person Syndrome, Striatonigral Degeneration, Stroke, Sturge-Weber Syndrome, SUNCT Headache, Syncope, Syphilitic Spinal Sclerosis, Syringomyelia, Tabes Dorsalis, Tardive Dyskinesia, Tarlov Cysts, Tay-Sachs Disease, Temporal Arteritis, Tethered Spinal Cord Syndrome, Thomsen's Myotonia, Thoracic Outlet Syndrome, Thyrotoxic Myopathy, Tinnitus, Todd's Paralysis, Tourette Syndrome, Transient Ischemic Attack, Transmissible Spongiform Encephalopathies, Transverse Myelitis, Traumatic Brain Injury, Tremor, Trigeminal Neuralgia, Tropical Spastic Paraparesis, Troyer Syndrome, Tuberous Sclerosis, Vasculitis including Temporal Arteritis, Von Economo's Disease, Von Hippel-Lindau Disease (VHL), Von Recklinghausen's Disease, Wallenberg's Syndrome, Werdnig-Hoffman Disease, Wernicke-Korsakoff Syndrome, West Syndrome, Whiplash, Whipple's Disease, Williams Syndrome, Wilson's Disease, Wolman's Disease, X-Linked Spinal and Bulbar Muscular Atrophy, and Zellweger Syndrome.

In various embodiments, the autoimmune disease or disorder can be any one of arthritis, including rheumatoid arthritis, acute arthritis, chronic rheumatoid arthritis, gout or gouty arthritis, acute gouty arthritis, acute immunological arthritis, chronic inflammatory arthritis, degenerative arthritis, type II collagen-induced arthritis, infectious arthritis, Lyme arthritis, proliferative arthritis, psoriatic arthritis, Still's disease, vertebral arthritis, juvenile-onset rheumatoid arthritis, osteoarthritis, arthritis deformans, polyarthritis chronica primaria, reactive arthritis, and ankylosing spondylitis; inflammatory hyperproliferative skin diseases; psoriasis, such as plaque psoriasis, pustular psoriasis, and psoriasis of the nails; atopy, including atopic diseases such as hay fever and Job's syndrome; dermatitis, including contact dermatitis, chronic contact dermatitis, exfoliative dermatitis, allergic dermatitis, allergic contact dermatitis, dermatitis herpetiformis, nummular dermatitis, seborrheic dermatitis, non-specific dermatitis, primary irritant contact dermatitis, and atopic dermatitis; x-linked hyper IgM syndrome; allergic intraocular inflammatory diseases; urticaria, such as chronic allergic urticaria, chronic idiopathic urticaria, and chronic autoimmune urticaria; myositis; polymyositis/dermatomyositis; juvenile dermatomyositis; toxic epidermal necrolysis; scleroderma, including systemic scleroderma; sclerosis, such as systemic sclerosis, multiple sclerosis (MS), spino-optical MS, primary progressive MS (PPMS), relapsing remitting MS (RRMS), progressive systemic sclerosis, atherosclerosis, arteriosclerosis, sclerosis disseminata, and ataxic sclerosis; neuromyelitis optica (NMO); inflammatory bowel disease (IBD), including Crohn's disease, autoimmune-mediated gastrointestinal diseases, colitis, ulcerative colitis, colitis ulcerosa, microscopic colitis, collagenous colitis, colitis polyposa, necrotizing enterocolitis, transmural colitis, and autoimmune inflammatory bowel disease; bowel inflammation; pyoderma gangrenosum; erythema nodosum; primary sclerosing cholangitis; respiratory distress syndrome, including adult or acute respiratory distress syndrome (ARDS); meningitis; inflammation of all or part of the uvea; iritis; choroiditis; an autoimmune hematological disorder; rheumatoid spondylitis; rheumatoid synovitis; hereditary angioedema; cranial nerve damage, as in meningitis; herpes gestationis; pemphigoid gestationis; pruritis scroti; autoimmune premature ovarian failure; sudden hearing loss due to an autoimmune condition; IgE-mediated diseases, such as anaphylaxis and allergic and atopic rhinitis; encephalitis, such as Rasmussen's encephalitis and limbic and/or brainstem encephalitis; uveitis, such as anterior uveitis, acute anterior uveitis, granulomatous uveitis, nongranulomatous uveitis, phacoantigenic uveitis, posterior uveitis, or autoimmune uveitis; glomerulonephritis (GN) with and without nephrotic syndrome, such as chronic or acute glomerulonephritis, primary GN, immune-mediated GN, membranous GN (membranous nephropathy), idiopathic membranous GN or idiopathic membranous nephropathy, membrano- or membranous proliferative GN (MPGN), including Type I and Type II, and rapidly progressive GN; proliferative nephritis; autoimmune polyglandular endocrine failure; balanitis, including balanitis circumscripta plasmacellularis; balanoposthitis; erythema annulare centrifugum; erythema dyschromicum perstans; eythema multiform; granuloma annulare; lichen nitidus; lichen sclerosus et atrophicus; lichen simplex chronicus; lichen spinulosus; lichen planus; lamellar ichthyosis; epidermolytic hyperkeratosis; premalignant keratosis; pyoderma gangrenosum; allergic conditions and responses; allergic reaction; eczema, including allergic or atopic eczema, asteatotic eczema, dyshidrotic eczema, and vesicular palmoplantar eczema; asthma, such as asthma bronchiale, bronchial asthma, and auto-immune asthma; conditions involving infiltration of T cells and chronic inflammatory responses; immune reactions against foreign antigens such as fetal A-B-O blood groups during pregnancy; chronic pulmonary inflammatory disease; autoimmune myocarditis; leukocyte adhesion deficiency; lupus, including lupus nephritis, lupus cerebritis, pediatric lupus, non-renal lupus, extra-renal lupus, discoid lupus and discoid lupus erythematosus, alopecia lupus, systemic lupus erythematosus (SLE), cutaneous SLE, subacute cutaneous SLE, neonatal lupus syndrome (NLE), and lupus erythematosus disseminatus; juvenile onset (Type I) diabetes mellitus, including pediatric insulin-dependent diabetes mellitus (IDDM), adult onset diabetes mellitus (Type II diabetes), autoimmune diabetes, idiopathic diabetes insipidus, diabetic retinopathy, diabetic nephropathy, and diabetic large-artery disorder; immune responses associated with acute and delayed hypersensitivity mediated by cytokines and T-lymphocytes; tuberculosis; sarcoidosis; granulomatosis, including lymphomatoid granulomatosis; Wegener's granulomatosis; agranulocytosis; vasculitides, including vasculitis, large-vessel vasculitis, polymyalgia rheumatica and giant-cell (Takayasu's) arteritis, medium-vessel vasculitis, Kawasaki's disease, polyarteritis nodosa/periarteritis nodosa, microscopic polyarteritis, immunovasculitis, CNS vasculitis, cutaneous vasculitis, hypersensitivity vasculitis, necrotizing vasculitis, systemic necrotizing vasculitis, ANCA-associated vasculitis, Churg-Strauss vasculitis or syndrome (CSS), and ANCA-associated small-vessel vasculitis; temporal arteritis; aplastic anemia; autoimmune aplastic anemia; Coombs positive anemia; Diamond Blackfan anemia; hemolytic anemia or immune hemolytic anemia, including autoimmune hemolytic anemia (AIHA), pernicious anemia (anemia perniciosa); Addison's disease; pure red cell anemia or aplasia (PRCA); Factor VIII deficiency; hemophilia A; autoimmune neutropenia; pancytopenia; leukopenia; diseases involving leukocyte diapedesis; CNS inflammatory disorders; multiple organ injury syndrome, such as those secondary to septicemia, trauma or hemorrhage; antigen-antibody complex-mediated diseases; anti-glomerular basement membrane disease; anti-phospholipid antibody syndrome; allergic neuritis; Behcet's disease/syndrome; Castleman's syndrome; Goodpasture's syndrome; Reynaud's syndrome; Sjogren's syndrome; Stevens-Johnson syndrome; pemphigoid, such as pemphigoid bullous and skin pemphigoid, pemphigus, pemphigus vulgaris, pemphigus foliaceus, pemphigus mucus-membrane pemphigoid, and pemphigus erythematosus; autoimmune polyendocrinopathies; Reiter's disease or syndrome; thermal injury; preeclampsia; an immune complex disorder, such as immune complex nephritis, and antibody-mediated nephritis; polyneuropathies; chronic neuropathy, such as IgM polyneuropathies and IgM-mediated neuropathy; thrombocytopenia (as developed by myocardial infarction patients, for example), including thrombotic thrombocytopenic purpura (TTP), post-transfusion purpura (PTP), heparin-induced thrombocytopenia, autoimmune or immune-mediated thrombocytopenia, idiopathic thrombocytopenic purpura (ITP), and chronic or acute ITP; scleritis, such as idiopathic cerato-scleritis, and episcleritis; autoimmune disease of the testis and ovary including, autoimmune orchitis and oophoritis; primary hypothyroidism; hypoparathyroidism; autoimmune endocrine diseases, including thyroiditis, autoimmune thyroiditis, Hashimoto's disease, chronic thyroiditis (Hashimoto's thyroiditis), or subacute thyroiditis, autoimmune thyroid disease, idiopathic hypothyroidism, Grave's disease, polyglandular syndromes, autoimmune polyglandular syndromes, and polyglandular endocrinopathy syndromes; paraneoplastic syndromes, including neurologic paraneoplastic syndromes; Lambert-Eaton myasthenic syndrome or Eaton-Lambert syndrome; stiff-man or stiff-person syndrome; encephalomyelitis, such as allergic encephalomyelitis, encephalomyelitis allergica, and experimental allergic encephalomyelitis (EAE); myasthenia gravis, such as thymoma-associated myasthenia gravis; cerebellar degeneration; neuromyotonia; opsoclonus or opsoclonus myoclonus syndrome (OMS); sensory neuropathy; multifocal motor neuropathy; Sheehan's syndrome; hepatitis, including autoimmune hepatitis, chronic hepatitis, lupoid hepatitis, giant-cell hepatitis, chronic active hepatitis, and autoimmune chronic active hepatitis; lymphoid interstitial pneumonitis (LIP); bronchiolitis obliterans (non-transplant) vs NSIP; Guillain-Barre syndrome; Berger's disease (IgA nephropathy); idiopathic IgA nephropathy; linear IgA dermatosis; acute febrile neutrophilic dermatosis; subcorneal pustular dermatosis; transient acantholytic dermatosis; cirrhosis, such as primary biliary cirrhosis and pneumonocirrhosis; autoimmune enteropathy syndrome; Celiac or Coeliac disease; celiac sprue (gluten enteropathy); refractory sprue; idiopathic sprue; cryoglobulinemia; amylotrophic lateral sclerosis (ALS; Lou Gehrig's disease); coronary artery disease; autoimmune ear disease, such as autoimmune inner ear disease (AIED); autoimmune hearing loss; polychondritis, such as refractory or relapsed or relapsing polychondritis; pulmonary alveolar proteinosis; Cogan's syndrome/nonsyphilitic interstitial keratitis; Bell's palsy; Sweet's disease/syndrome; rosacea autoimmune; zoster-associated pain; amyloidosis; a non-cancerous lymphocytosis; a primary lymphocytosis, including monoclonal B cell lymphocytosis (e.g., benign monoclonal gammopathy and monoclonal gammopathy of undetermined significance, MGUS); peripheral neuropathy; channelopathies, such as epilepsy, migraine, arrhythmia, muscular disorders, deafness, blindness, periodic paralysis, and channelopathies of the CNS; autism; inflammatory myopathy; focal or segmental or focal segmental glomerulosclerosis (FSGS); endocrine opthalmopathy; uveoretinitis; chorioretinitis; autoimmune hepatological disorder; fibromyalgia; multiple endocrine failure; Schmidt's syndrome; adrenalitis; gastric atrophy; presenile dementia; demyelinating diseases, such as autoimmune demyelinating diseases and chronic inflammatory demyelinating polyneuropathy; Dressler's syndrome; alopecia areata; alopecia totalis; CREST syndrome (calcinosis, Raynaud's phenomenon, esophageal dysmotility, sclerodactyly, and telangiectasia); male and female autoimmune infertility (e.g., due to anti-spermatozoan antibodies); mixed connective tissue disease; Chagas' disease; rheumatic fever; recurrent abortion; farmer's lung; erythema multiforme; post-cardiotomy syndrome; Cushing's syndrome; bird-fancier's lung; allergic granulomatous angiitis; benign lymphocytic angiitis; Alport's syndrome; alveolitis, such as allergic alveolitis and fibrosing alveolitis; interstitial lung disease; transfusion reaction; leprosy; malaria; Samter's syndrome; Caplan's syndrome; endocarditis; endomyocardial fibrosis; diffuse interstitial pulmonary fibrosis; interstitial lung fibrosis; pulmonary fibrosis; idiopathic pulmonary fibrosis; cystic fibrosis; endophthalmitis; erythema elevatum et diutinum; erythroblastosis fetalis; eosinophilic fasciitis; Shulman's syndrome; Felty's syndrome; flariasis; cyclitis, such as chronic cyclitis, heterochronic cyclitis, iridocyclitis (acute or chronic), or Fuch's cyclitis; Henoch-Schonlein purpura; sepsis; endotoxemia; pancreatitis; thyroxicosis; Evan's syndrome; autoimmune gonadal failure; Sydenham's chorea; post-streptococcal nephritis; thromboangitis ubiterans; thyrotoxicosis; tabes dorsalis; choroiditis; giant-cell polymyalgia; chronic hypersensitivity pneumonitis; keratoconjunctivitis sicca; epidemic keratoconjunctivitis; idiopathic nephritic syndrome; minimal change nephropathy; benign familial and ischemia-reperfusion injury; transplant organ reperfusion; retinal autoimmunity; joint inflammation; bronchitis; chronic obstructive airway/pulmonary disease; silicosis; aphthae; aphthous stomatitis; arteriosclerotic disorders; aspermiogenese; autoimmune hemolysis; Boeck's disease; cryoglobulinemia; Dupuytren's contracture; endophthalmia phacoanaphylactica; enteritis allergica; erythema nodo sum leprosum; idiopathic facial paralysis; febris rheumatica; Hamman-Rich's disease; sensoneural hearing loss; haemoglobinuria paroxysmatica; hypogonadism; ileitis regionalis; leucopenia; mononucleosis infectiosa; traverse myelitis; primary idiopathic myxedema; nephrosis; ophthalmia symphatica; orchitis granulomatosa; pancreatitis; polyradiculitis acuta; pyoderma gangrenosum; Quervain's thyreoiditis; acquired splenic atrophy; non-malignant thymoma; vitiligo; toxic-shock syndrome; food poisoning; conditions involving infiltration of T cells; leukocyte-adhesion deficiency; immune responses associated with acute and delayed hypersensitivity mediated by cytokines and T-lymphocytes; diseases involving leukocyte diapedesis; multiple organ injury syndrome; antigen-antibody complex-mediated diseases; antiglomerular basement membrane disease; allergic neuritis; autoimmune polyendocrinopathies; oophoritis; primary myxedema; autoimmune atrophic gastritis; sympathetic ophthalmia; rheumatic diseases; mixed connective tissue disease; nephrotic syndrome; insulitis; polyendocrine failure; autoimmune polyglandular syndrome type I; adult-onset idiopathic hypoparathyroidism (AOIH); cardiomyopathy such as dilated cardiomyopathy; epidermolisis bullosa acquisita (EBA); hemochromatosis; myocarditis; nephrotic syndrome; primary sclerosing cholangitis; purulent or nonpurulent sinusitis; acute or chronic sinusitis; ethmoid, frontal, maxillary, or sphenoid sinusitis; an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, or granulomas containing eosinophils; anaphylaxis; seronegative spondyloarthritides; polyendocrine autoimmune disease; sclerosing cholangitis; chronic mucocutaneous candidiasis; Bruton's syndrome; transient hypogammaglobulinemia of infancy; Wiskott-Aldrich syndrome; ataxia telangiectasia syndrome; angiectasis; autoimmune disorders associated with collagen disease, rheumatism, neurological disease, lymphadenitis, reduction in blood pressure response, vascular dysfunction, tissue injury, cardiovascular ischemia, hyperalgesia, renal ischemia, cerebral ischemia, and disease accompanying vascularization; allergic hypersensitivity disorders; glomerulonephritides; reperfusion injury; ischemic reperfusion disorder; reperfusion injury of myocardial or other tissues; lymphomatous tracheobronchitis; inflammatory dermatoses; dermatoses with acute inflammatory components; multiple organ failure; bullous diseases; renal cortical necrosis; acute purulent meningitis or other central nervous system inflammatory disorders; ocular and orbital inflammatory disorders; granulocyte transfusion-associated syndromes; cytokine-induced toxicity; narcolepsy; acute serious inflammation; chronic intractable inflammation; pyelitis; endarterial hyperplasia; peptic ulcer; valvulitis; and endometriosis. In particular embodiments, the autoimmune disorder in the subject can include one or more of: systemic lupus erythematosus (SLE), lupus nephritis, chronic graft versus host disease (cGVHD), rheumatoid arthritis (RA), Sjogren's syndrome, vitiligo, inflammatory bowed disease, and Crohn's Disease. In particular embodiments, the autoimmune disorder is systemic lupus erythematosus (SLE). In particular embodiments, the autoimmune disorder is rheumatoid arthritis.

Exemplary metabolic disorders include, for example, diabetes, insulin resistance, lysosomal storage disorders (e.g., Gauchers disease, Krabbe disease, Niemann Pick disease types A and B, multiple sclerosis, Fabry's disease, Tay Sachs disease, and Sandhoff Variant A, B), obesity, cardiovascular disease, and dyslipidemia. Other exemplary metabolic disorders include, for example, 17-alpha-hydroxylase deficiency, 17-beta hydroxysteroid dehydrogenase 3 deficiency, 18 hydroxylase deficiency, 2-hydroxyglutaric aciduria, 2-methylbutyryl-CoA dehydrogenase deficiency, 3-alpha hydroxyacyl-CoA dehydrogenase deficiency, 3-hydroxyisobutyric aciduria, 3-methylcrotonyl-CoA carboxylase deficiency, 3-methylglutaconyl-CoA hydratase deficiency (AUH defect), 5-oxoprolinase deficiency, 6-pyruvoyl-tetrahydropterin synthase deficiency, abdominal obesity metabolic syndrome, abetalipoproteinemia, acatalasemia, aceruloplasminemia, acetyl CoA acetyltransferase 2 deficiency, acetyl-carnitine deficiency, acrodermatitis enteropathica, adenine phosphoribosyltransferase deficiency, adenosine deaminase deficiency, adenosine monophosphate deaminase 1 deficiency, adenylosuccinase deficiency, adrenomyeloneuropathy, adult polyglucosan body disease, albinism deafness syndrome, alkaptonuria, Alpers syndrome, alpha-1 antitrypsin deficiency, alpha-ketoglutarate dehydrogenase deficiency, alpha-mannosidosis, aminoacylase 1 deficiency, anemia sideroblastic and spinocerebellar ataxia, arginase deficiency, argininosuccinic aciduria, aromatic L-amino acid decarboxylase deficiency, arthrogryposis renal dysfunction cholestasis syndrome, Arts syndrome, aspartylglycosaminuria, atypical Gaucher disease due to saposin C deficiency, autoimmune polyglandular syndrome type 2, autosomal dominant optic atrophy and cataract, autosomal erythropoietic protoporphyria, autosomal recessive spastic ataxia 4, Barth syndrome, Bartter syndrome, Bartter syndrome antenatal type 1, Bartter syndrome antenatal type 2, Bartter syndrome type 3, Bartter syndrome type 4, Beta ketothiolase deficiency, biotinidase deficiency, Bjornstad syndrome, carbamoyl phosphate synthetase 1 deficiency, carnitine palmitoyl transferase 1A deficiency, carnitine-acylcarnitine translocase deficiency, carnosinemia, central diabetes insipidus, cerebral folate deficiency, cerebrotendinous xanthomatosis, ceroid lipofuscinosis neuronal 1, Chanarin-Dorfman syndrome, Chediak-Higashi syndrome, childhood hypophosphatasia, cholesteryl ester storage disease, chondrocalcinosisc, chylomicron retention disease, citrulline transport defect, congenital bile acid synthesis defect, type 2, Crigler Najjar syndrome, cytochrome c oxidase deficiency, D-2-hydroxyglutaric aciduria, D-bifunctional protein deficiency, D-glycericacidemia, Danon disease, dicarboxylic aminoaciduria, dihydropteridine reductase deficiency, dihydropyrimidinase deficiency, diabetes insipidus, dopamine beta hydroxylase deficiency, Dowling-Degos disease, erythropoietic uroporphyria associated with myeloid malignancy, Familial chylomicronemia syndrome, Familial HDL deficiency, Familial hypocalciuric hypercalcemia type 1, Familial hypocalciuric hypercalcemia type 2, Familial hypocalciuric hypercalcemia type 3, Familial LCAT deficiency, Familial partial lipodystrophy type 2, Fanconi Bickel syndrome, Farber disease, fructose-1,6-bisphosphatase deficiency, gamma-cystathionase deficiency, Gaucher disease, Gilbert syndrome, Gitelman syndrome, glucose transporter type 1 deficiency syndrome, glutamine deficiency, congenital, Glutaric acidemia, glutathione synthetase deficiency, glycine N-methyltransferase deficiency, Glycogen storage disease hepatic lipase deficiency, homocysteinemia, Hurler syndrome, hyperglycerolemia, Imerslund-Grasbeck syndrome, iminoglycinuria, infantile neuroaxonal dystrophy, Kearns-Sayre syndrome, Krabbe disease, lactate dehydrogenase deficiency, Lesch Nyhan syndrome, Menkes disease, methionine adenosyltransferase deficiency, mitochondrial complex deficiency, muscular phosphorylase kinase deficiency, neuronal ceroid lipofuscinosis, Niemann-Pick disease type A, Niemann-Pick disease type B, Niemann-Pick disease type C1, Niemann-Pick disease type C2, ornithine transcarbamylase deficiency, Pearson syndrome, Perrault syndrome, phosphoribosylpyrophosphate synthetase superactivity, primary carnitine deficiency, hyperoxaluria, purine nucleoside phosphorylase deficiency, pyruvate carboxylase deficiency, pyruvate dehydrogenase complex deficiency, pyruvate dehydrogenase phosphatase deficiency, yruvate kinase deficiency, Refsum disease, diabetes mellitus, Scheie syndrome, Sengers syndrome, Sialidosis Sjogren-Larsson syndrome, Tay-Sachs disease, transcobalamin 1 deficiency, trehalase deficiency, Walker-Warburg syndrome, Wilson disease, Wolfram syndrome, and Wolman disease.

Computer Implementation

The methods of the invention, including the methods of performing an intra-individual analysis to determine a presence or absence of a health condition, are, in some embodiments, performed on one or more computers. In particular embodiments, the step of performing an intra-individual analysis (e.g., step 130 shown in FIG. 1) is performed on one or more computers. In particular embodiments, the steps of performing an assay (e.g., assay 120A and/or assay 120B shown in FIG. 1) are not performed on one or more computers.

In various embodiments, the performance of the intra-individual analysis can be implemented in hardware or software, or a combination of both. In one embodiment of the invention, a machine-readable storage medium is provided, the medium comprising a data storage material encoded with machine readable data which, when using a machine programmed with instructions for using said data, is capable of displaying data and results of the intra-individual analysis. The invention can be implemented in computer programs executing on programmable computers, comprising a processor, a data storage system (including volatile and non-volatile memory and/or storage elements), a graphics adapter, a pointing device, a network adapter, at least one input device, and at least one output device. A display is coupled to the graphics adapter. Program code is applied to input data to perform the functions described above and generate output information. The output information is applied to one or more output devices, in known fashion. The computer can be, for example, a personal computer, microcomputer, or workstation of conventional design.

Each program can be implemented in a high level procedural or object oriented programming language to communicate with a computer system. However, the programs can be implemented in assembly or machine language, if desired. In any case, the language can be a compiled or interpreted language. Each such computer program is preferably stored on a storage media or device (e.g., ROM or magnetic diskette) readable by a general or special purpose programmable computer, for configuring and operating the computer when the storage media or device is read by the computer to perform the procedures described herein. The system can also be considered to be implemented as a computer-readable storage medium, configured with a computer program, where the storage medium so configured causes a computer to operate in a specific and predefined manner to perform the functions described herein.

The signature patterns and databases thereof can be provided in a variety of media to facilitate their use. “Media” refers to a manufacture that contains the signature pattern information of the present invention. The databases of the present invention can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure can be chosen, based on the means used to access the stored information. A variety of data processor programs and formats can be used for storage, e.g. word processing text file, database format, etc.

In some embodiments, the methods of the invention, including methods of intra-individual analysis, are performed on one or more computers in a distributed computing system environment (e.g., in a cloud computing environment). In this description, “cloud computing” is defined as a model for enabling on-demand network access to a shared set of configurable computing resources. Cloud computing can be employed to offer on-demand access to the shared set of configurable computing resources. The shared set of configurable computing resources can be rapidly provisioned via virtualization and released with low management effort or service provider interaction, and then scaled accordingly. A cloud-computing model can be composed of various characteristics such as, for example, on-demand self-service, broad network access, resource pooling, rapid elasticity, measured service, and so forth. A cloud-computing model can also expose various service models, such as, for example, Software as a Service (“SaaS”), Platform as a Service (“PaaS”), and Infrastructure as a Service (“IaaS”). A cloud-computing model can also be deployed using different deployment models such as private cloud, community cloud, public cloud, hybrid cloud, and so forth. In this description and in the claims, a “cloud-computing environment” is an environment in which cloud computing is employed.

### Example Computer

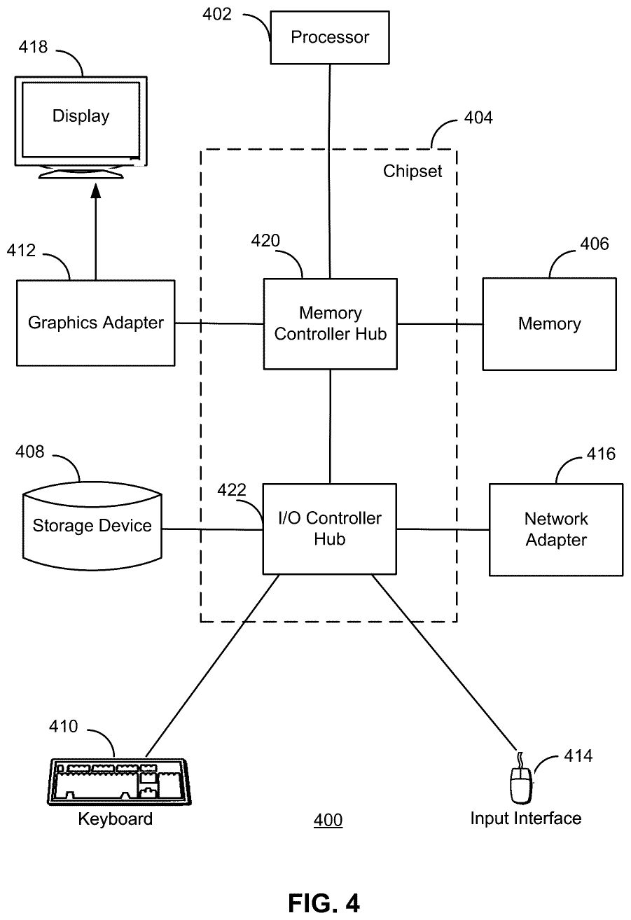

FIG. 4 illustrates an example computer for implementing the entities shown in FIGS. 1, 2A, 2B, and 3. In particular embodiments, the example computer 400 can represent computational system 202 described in FIG. 2. The computer 400 includes at least one processor 402 coupled to a chipset 404. The chipset 404 includes a memory controller hub 420 and an input/output (I/O) controller hub 422. A memory 406 and a graphics adapter 412 are coupled to the memory controller hub 420, and a display 418 is coupled to the graphics adapter 412. A storage device 408, an input device 414, and network adapter 416 are coupled to the I/O controller hub 422. Other embodiments of the computer 400 have different architectures.

The storage device 408 is a non-transitory computer-readable storage medium such as a hard drive, compact disk read-only memory (CD-ROM), DVD, or a solid-state memory device. The memory 406 holds instructions and data used by the processor 402. The input interface 414 is a touch-screen interface, a mouse, track ball, or other type of pointing device, a keyboard, or some combination thereof, and is used to input data into the computer 400. In some embodiments, the computer 400 may be configured to receive input (e.g., commands) from the input interface 414 via gestures from the user. The graphics adapter 412 displays images and other information on the display 418. The network adapter 416 couples the computer 400 to one or more computer networks.

The computer 400 is adapted to execute computer program modules for providing functionality described herein. As used herein, the term “module” refers to computer program logic used to provide the specified functionality. Thus, a module can be implemented in hardware, firmware, and/or software. In one embodiment, program modules are stored on the storage device 408, loaded into the memory 406, and executed by the processor 402. A module can be implemented as computer program code processed by the processing system(s) of one or more computers. Computer program code includes computer-executable instructions and/or computer-interpreted instructions, such as program modules, which instructions are processed by a processing system of a computer. Generally, such instructions define routines, programs, objects, components, data structures, and so on, that, when processed by a processing system, instruct the processing system to perform operations on data or configure the processor or computer to implement various components or data structures in computer storage. A data structure is defined in a computer program and specifies how data is organized in computer storage, such as in a memory device or a storage device, so that the data can accessed, manipulated, and stored by a processing system of a computer.

The types of computers 400 used can vary depending upon the embodiment and the processing power required by the entity. For example, the health condition system 220 can run in a single computer 400 or multiple computers 400 communicating with each other through a network such as in a server farm. The computers 400 can lack some of the components described above, such as graphics adapters 412, and displays 418.

Kit Implementation

Also disclosed herein are kits for performing an intra-individual analysis. Such kits can include equipment to draw a sample from a patient. For example, kits can include syringes and/or needles for obtaining a sample from a patient. Kits can include detection reagents for determining sequence information using the sample obtained from the patient.

For example, detection reagents can be a set of primers that, when combined with the sample, allows detection of statuses for a plurality of sites in nucleic acids in a sample. In particular embodiments, the detection reagents enable detection of methylated or unmethylated target sites (e.g., methylated or unmethylated informative CpGs including one or more CpG islands or portions of CpG islands shown in Tables 1-4). For example, the detection reagents may be primers that target specific known sequences of target sites, thereby enabling nucleic acid amplification of the target sites. Thus, the use of the detection reagents results in generation of methylation information of the patient corresponding to the target sites. In various embodiments, the detection reagents can be used to detect statuses for a plurality of sites in different nucleic acids in different samples. For example, the kit may include detection reagents for detecting statuses for a plurality of sites in target nucleic acids in a sample and/or a plurality of sites in reference nucleic acids in a different sample.

A kit can include instructions for use of one or more sets of detection reagents. For example, a kit can include instructions for performing at least one detection assay such as a nucleic acid amplification assay (e.g., polymerase chain reaction assay including any of real-time PCR assays, quantitative real-time PCR (qPCR) assays, allele-specific PCR assays, and reverse-transcription PCR assays), nucleic acid sequencing (e.g., targeted gene sequencing, targeted amplicon sequencing, whole genome sequencing, or whole genome bisulfite sequencing), hybrid capture, an immunoassay, a protein-binding assay, an antibody-based assay, an antigen-binding protein-based assay, a protein-based array, an enzyme-linked immunosorbent assay (ELISA), reporter assays, flow cytometry, a protein array, a blot, a Western blot, nephelometry, turbidimetry, chromatography, NMR, mass spectrometry, LC-MS, UPLC-MS/MS, enzymatic activity, proximity extension assay, and an immunoassay selected from RIA, immunofluorescence, immunochemiluminescence, immunoelectrochemiluminescence, immunoelectrophoretic, a competitive immunoassay, and immunoprecipitation.

Kits can further include instructions for accessing computer program instructions stored on a computer storage medium. In various embodiments, the computer program instructions, when executed by a processor of a computer system, cause the processor to perform an intra-individual analysis. For example, kits can include instructions that, when executed by a processor of a computer system, cause the processor to combine sequence information from target nucleic acids and sequence information from reference nucleic acids to generate a signal informative of the health condition. The kit can further include instructions that, when executed by a processor of a computer system, cause the processor to analyze the signal informative of the health condition to predict whether the individual has a presence or absence of the health condition.

In various embodiments, the kits include instructions for practicing the methods disclosed herein (e.g., performing an assay and/or performing an intra-individual analysis). These instructions can be present in the kits in a variety of forms, one or more of which can be present in the kit. One form in which these instructions can be present is as printed information on a suitable medium or substrate, e.g., a piece or pieces of paper on which the information is printed, in the packaging of the kit, in a package insert, etc. Yet another means would be a computer readable medium, e.g., diskette, CD, hard-drive, network data storage, etc., on which the information has been recorded. Yet another means that can be present is a website address which can be used via the internet to access the information at a removed site. Any convenient means can be present in the kits.

Systems

Further disclosed herein are systems for performing an intra-individual analysis. In various embodiments, such a system can include one or more sets of detection reagents for determining sequence information from target nucleic acids and/or reference nucleic acids using one or more samples obtained from the patient, an apparatus configured to receive a mixture of the one or more sets of detection reagents and the one or more samples obtained from the patient to generate sequence information from the target nucleic acids and reference nucleic acids for the patient, and a computer system communicatively coupled to the apparatus to obtain the sequence information from the target nucleic acids and reference nucleic acids and to perform an intra-individual analysis.

The one or more sets of detection reagents enable the determination of sequence information using the sample obtained from the patient. For example, detection reagents can be a set of primers that, when combined with the sample, allows detection of a plurality of sites in nucleic acids, such as target nucleic acids or reference nucleic acids, in the sample. In particular embodiments, the detection reagents enable detection of methylated or methylated target sites (e.g., methylated or unmethylated informative CpGs including one or more CpG islands or portions of CpG islands shown in Tables 1-4).

The apparatus is configured to determine the sequence information from a mixture of the detection reagents and sample. For example, the apparatus can be configured to perform one or more of a nucleic acid amplification assay (e.g., polymerase chain reaction assay), nucleic acid sequencing (e.g., targeted gene sequencing, whole genome sequencing, or whole genome bisulfite sequencing), or hybrid capture to determine sequence information. Such apparatuses can be example assay apparatus 205A, assay apparatus 205B, and/or assay apparatus 205C included as part of the health condition system 200 (see FIG. 2).

The mixture of the detection reagents and sample may be presented to the apparatus through various conduits, examples of which include wells of a well plate (e.g., 96 well plate), a vial, a tube, and integrated fluidic circuits. As such, the apparatus may have an opening (e.g., a slot, a cavity, an opening, a sliding tray) that can receive the container including the reagent test sample mixture and perform a reading. Examples of an apparatus include one or more of a sequencer, an incubator, plate reader (e.g., a luminescent plate reader, absorbance plate reader, fluorescence plate reader), a spectrometer, or a spectrophotometer.

The computer system, such as example computer 400 described in FIG. 4, communicates with the apparatus to receive the methylation information. The computer system performs an in silico intra-individual analysis to determine whether a health condition is present in the patient.

### EXAMPLES

Below are examples of specific embodiments for carrying out the present invention. The examples are offered for illustrative purposes only and are not intended to limit the scope of the present invention in any way. Efforts have been made to ensure accuracy with respect to numbers used (e.g., percentages, etc.), but some experimental error and deviation should be allowed for.

### Example 1: Example Samples and Assays for Conducting an Intra-Individual Analysis

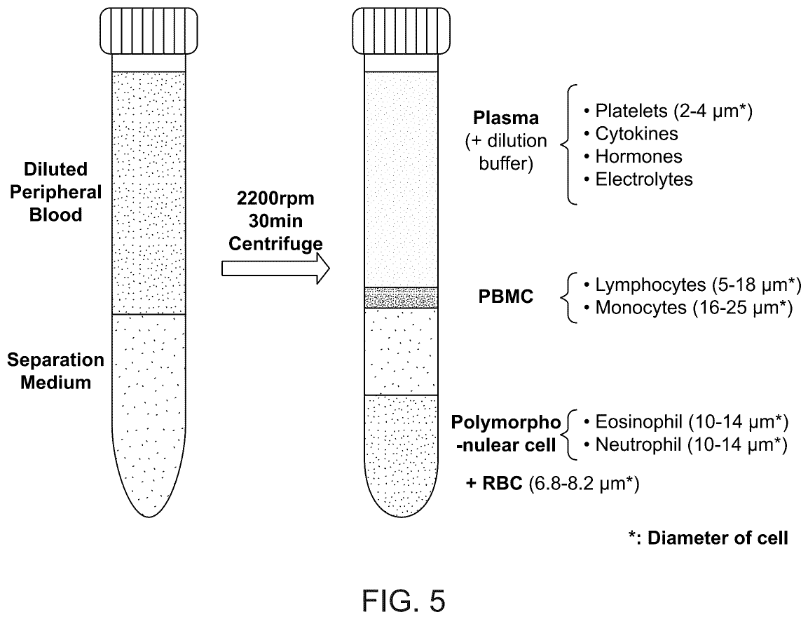

Blood samples are obtained from individuals. FIG. 5 shows an example sample from which target nucleic acids and reference nucleic acids are obtained. Shown on the left in FIG. 5 is a tube of blood obtained from an individual, the tube including diluted peripheral blood of the individual and separation medium. The tube undergoes centrifugation to separate different components of the diluted peripheral blood. For example, at a speed of 2200 rpm, the diluted peripheral blood is fractionated into plasma (including platelets, cytokines, hormones, and electrolytes), peripheral blood mononuclear cells (PBMCs), the separation medium, and polymorphonuclear cells. Here, target nucleic acids in the form of cell free DNA is found in the plasma whereas reference nucleic acids in the form of cellular genomic DNA is found in PBMCs.

Examples of an assay for generating sequence information from the target nucleic acids and the reference nucleic acids include but are not limited to Allele-specific PCR assays, Next Generation Sequencing assays, such as target enrichment technologies, targeted amplicon sequencing technologies, and whole genome sequencing.

An example protocol of an Allele-specific Real-Time PCR assay is as follows:
1. This assay runs all cfDNA samples in triplicate with 2 ng input in 5 uL for the reference and hypermethylation assays.
2. Combine 900 nmol/L unspecific primer(s), 100 nmol/L target probe(s), 2× polymerase enzyme(s), 2× dNTPs, 2× passive reference dyes, 10 uL water and 2 ng sample DNA at a pre-specified reaction volume as the reference control assay.
3. Combine 450 nmol/L allele-specific primer(s), 100 nmol/L target probe(s), 2× polymerase enzyme(s), 2× dNTPs, 2× passive reference dyes, 10 uL water and 2 ng sample DNA at a pre-specified reaction volume as the mutation assay.
4. Mix each reaction 10× and centrifuge to collect volume at the bottom of the well or tube.
5. Run the real-time PCR on a calibrated Real-Time PCR system under the following conditions: (1) 95° C. for 10 minutes followed by (2) 50 cycles of 90° C. for 15 seconds and 60° C. for 1 minute with fluorescence detection using FAM/VIC fluorophores.
6. Cycle threshold (Ct) values are recorded by the system and exported into an analysis program (e.g. Excel).
7. Average the Ct values between sample replicates for the reference and mutation assays.
8. Calculate the DCt between the sample average allele-specific Ct minus the sample average unspecific (reference) Ct.
9. Positive hypermethylation results are identified by the DCt cut off >3 cycles and will be compared to the patients individual PBMC natural signal.

An example protocol of an Allele-specific Real-Time PCR assay is as follows: Allele-specific real-time PCR can be performed by combining library from cfDNA with PCR reagents and primers specific for target sequences. The primers are designed to have single-base discrimination between tumor and non-tumor sequences. Perform real-time PCR (or digital PCR) for 30-50 cycles and monitor the output for signal via fluorescence from amplified target DNA or probe sequence. Cycle threshold values (Ct) are recorded and exported for analysis. The delta-Ct between negative control, positive control, and sample are calculated to determine presence or absence or absence of target tumor sequences. Slight modifications of this protocol will allow for end-point PCR detection of RNA or DNA of tumor sequences.

An example protocol of a next generation sequencing (NGS) Target Enrichment assay is as follows: The target specimen for library construction is dsDNA isolated from PBMCs. The dsDNA is first mechanically sheared by the Covaris instrument utilizing adaptive focused acoustics to a target insert size of 200 base pairs. Post-shearing, a solid-phase reversible immobilization (SPRI) selection is done to remove smaller DNA fragments remaining in solution. The fragmented DNA is then end-repaired and A-tailed (ERAT) to produce 5′-phosphorylated, 3′-dA-tailed dsDNA fragments. After ERAT, dsDNA unique dual index adapters with 3′-dTMP overhangs are then ligated to 3′-dA-tailed dsDNA fragments. Indices allow for sample multiplex for the downstream assay. Post-ligation, a solid-phase reversible immobilization (SPRI) selection is done to remove unwanted DNA fragments, excess adapters and molecules. PCR amplification is performed with a high-fidelity, low-bias polymerase at 10 cycles. Post-PCR, a SPRI selection is done to remove unwanted DNA fragments, excess primers, excess adapters and excess molecules. After library construction, the library quality and quantity are evaluated using the Agilent TapeStation and Qubit Fluorometer, respectively.

Libraries that pass quality control checks move forward to target enrichment through hybridization capture. Target enrichment by hybridization capture is defined as a positive selection strategy to enrich low abundance regions of interest from NGS libraries, allowing for more accurate sequencing analysis of these target regions. Indexed libraries are multi-plexed and hybridized to a custom, sequence specific, biotinylated probeset. The vast excess of probes drives their hybridization to complementary library fragments. The library fragment-biotinylated probe hybrid is pulled down by streptavidin beads, thereby capturing the target regions of interest. The streptavidin bead-bound library is sequentially washed with buffers to remove non-specifically associated library fragments. Following washes and recovery of captured libraries, samples are enriched for on target fragments and depleted for off-target fragments. Depletion of off-target fragments reduces overall library yield, requiring post-capture library amplification by PCR. The final amplified library is enriched for regions of interest. The hybrid captured library quality and quantity is evaluated using the Agilent TapeStation and Qubit Fluorometer, respectively. Additionally, the enrichment efficiency is evaluated using an iSeq Sequencing run and calculation of percent of reads within target enrichment panel. Measuring percent on-target is a good first approximation of target enrichment efficiency because the reads aligning to the target enrichment (bait) region indicate efficient hybridization and subsequent capture.

Target enriched libraries that pass quality control checks move forward to NovaSeq sequencing. Captured libraries with non-overlapping indices from library construction are pooled to multiplex for sequencing. Sequencing is completed on the NovaSeq 6000 instrument using paired end 150×150 base sequencing with a 10% PhiX spike-in. Sequencing data generated is then demultiplexed utilizing the assigned index, aligned to the human genome and trimmed to enrich for insert sample data only. This cleaned-up data is then processed through a quality pipeline to collapse duplicate reads and evaluate the sequencing data generated. Once the data is collapsed, the data is processed through a proprietary analysis pipeline to identify differences from the reference alignment (e.g. mutations, chemical modifications, etc.). A report is then generated with the specific signal informative for determining presence or absence of a health condition.

1-208. (canceled)

209. A method for determining a signal informative of a health condition from an individual, the method comprising:
obtaining target nucleic acids and reference nucleic acids from one or more samples from the individual;
generating sequence information from the target nucleic acids and sequence information from the reference nucleic acids; and
combining the sequence information from the target nucleic acids and the sequence information from the reference nucleic acids to generate the signal informative of the health condition.

210. The method of claim 209, wherein the health condition is a cancer.

211. The method of claim 209, wherein obtaining target nucleic acids and reference nucleic acids from one or more samples comprises obtaining the target nucleic acids and the reference nucleic acids from a single sample of any one of a blood sample, a stool sample, a urine sample, a mucous sample, or a saliva sample.

212. The method of claim 211, wherein obtaining target nucleic acids and reference nucleic acids comprises fractionating the single sample, wherein the target nucleic acids are obtained from a first fraction of the single sample, and wherein the reference nucleic acids are obtained from a second fraction of the single sample.

213. The method of claim 209, wherein the target nucleic acids comprise cell free DNA (cfDNA) and wherein the reference nucleic acids comprise genomic DNA from cells of the individual.

214. The method of claim 213, wherein the cells of the individual comprise peripheral blood mononuclear cells (PBMCs) or polymorphonuclear cells.

215. The method of claim 209, wherein combining the sequence information from the target nucleic acids and the sequence information from the reference nucleic acids comprises aligning the sequence information from the target nucleic acids and the sequence information from the reference nucleic acids; and determining a difference between the sequence information from the target nucleic acids and the sequence information from the reference nucleic acids.

216. The method of claim 209, wherein the sequence information from the target nucleic acids comprises methylation sequence information of the target nucleic acids.

217. The method of claim 209, wherein the sequence information from the target nucleic acids comprises phased sequencing information of the target nucleic acids derived from one of two or more sources.

218. The method of claim 217, wherein the phased sequence information from the target nucleic acids is generated by:
aligning sequence reads of target nucleic acids to long sequence reads of reference nucleic acids to determine two or more sources of the target nucleic acids, wherein the long sequence reads of reference nucleic acids comprise at least 500 bases; and
categorizing target nucleic acids derived from one of the two or more sources.

219. The method of claim 220, wherein the two or more sources comprise a maternal chromosome and a paternal chromosome.

220. The method of claim 209, wherein the sequence information of the target nucleic acids and the sequence information of the reference nucleic acids both comprise methylation statuses for a plurality of genomic sites.

221. The method of claim 220, wherein the plurality of genomic sites comprise one or more CpG islands or portions of CpG islands shown in Tables 1-4.

222. The method of claim 209, wherein generating sequence information from the target nucleic acids and sequence information from the reference nucleic acids comprises performing an assay, wherein the assay comprises one or more of
a. sequencing of target nucleic acids and/or reference nucleic acids via targeted sequencing, whole genome sequencing, or whole genome bisulfite sequencing;
b. shallow sequencing and/or deep sequencing;
c. a nucleic acid amplification assay; and
d. an assay that generates methylation information.

223. The method of claim 222, wherein performing the assay comprises performing both shallow sequencing and deep sequencing.

224. The method of claim 223, wherein performing both shallow sequencing and deep sequencing comprises:
performing shallow sequencing to generate sequence information from the reference nucleic acids; and
performing deep sequencing to generate sequence information from the target nucleic acids.

225. The method of claim 224, wherein performing shallow sequencing comprises generating less than less than 50 reads per base, less than 40 reads per base, less than 30 reads per base, less than 20 reads per base, less than 10 reads per base, less than 9 reads per base, less than 8 reads per base, less than 7 reads per base, less than 6 reads per base, or less than 5 reads per base.

226. The method of any claim 224, wherein performing deep sequencing comprises generating greater than 50 reads per base, greater than 60 reads per base, greater than 70 reads per base, greater than 80 reads per base, greater than 90 reads per base, greater than 100 reads per base, greater than 120 reads per base, greater than 140 reads per base, greater than 150 reads per base, greater than 170 reads per base, greater than 200 reads per base, greater than 225 reads per base, greater than 250 reads per base, greater than 300 reads per base, greater than 400 reads per base, or greater than 500 reads per base.

227. The method of claim 209, wherein combining the sequence information from the target nucleic acids and the sequence information from the reference nucleic acids comprises determining ratios of methylation levels amongst two or more genomic sites from the target nucleic acids.

228. The method of claim 227, wherein combining the sequence information from the target nucleic acids and the sequence information from the reference nucleic acids further comprises:
determining a difference between the sequence information from target nucleic acids and sequence information from reference nucleic acids to generate a signal that includes limited or no baseline signatures;
determining additional ratios of methylation levels amongst the two or more CpG sites from the signal that includes limited or no baseline signatures;
comparing the ratios of methylation levels amongst two or more CpG sites generated from target nucleic acids and the additional ratios of methylation levels amongst the two or more CpG sites generated from the signal that includes limited or no baseline signatures; and
generating a prediction of presence or absence of the health condition based on the comparison.
